(19) United States
(12) Patent Application Publication
(10) Pub. No.: US 2015/0190625 A1
(43) Pub. Date: Jul. 9, 2015

(54) Fluid Transfer Devices

(71) Applicant: CONCEPTOMED AS, Ballstad (NO)

(72) Inventors: Marius Andresen, Oslo (NO); Rolf Blomvâgnes, Rong (NO); Kevin Geers, Oslo (NO); Christian Mide, Ballstad (NO)

(21) Appl. No.: 14/418,605

(22) PCT Filed: Jul. 31, 2013
(86) PCT No.: PCT/EP2013/066135

§ 371 Date: Jan. 30, 2015

(30) Foreign Application Priority Data
Jul. 31, 2012 (EP) 12178732.9

(51) Int. Cl. A61M 39/10; A61M 5/32

(52) U.S. Cl. CPC A61M 39/1011; A61M 5/3293; A61M 2039/1005

(57) ABSTRACT

A fluid transfer device may include a fluid transfer tip that has a tapered friction fitting for a corresponding hub and an additional means for gripping a hub in a locked position when connected to the fluid transfer tip by the friction fitting. A disconnecting member is provided with a resilient bias that causes it to engage the gripping means and thereby assist in holding the hub in the locked position. The disconnecting member is moveable relative to the fluid transfer tip and against the resilient bias so as to release engagement of the gripping means, move the hub out of the locked position provided by the gripping means and release the hub from the friction fitting.

The present invention relates to the detachment of fluid-transferring devices from a corresponding hub, in particular to the removal of syringes, and especially when transferring fluid in a medical setting. The invention may find particular use in detaching a fluid-transferring device from a hub that is connected to a living subject to/from whom fluid is being transferred.

In a medical setting it may be necessary or desirable to transfer fluid to/from a subject for a variety of reasons. For example, a hub connected to a needle or other cannula may be used to draw blood from a vein or to infuse fluid substances i.e. intravenous (IV) therapy. A drip is one type of IV therapy. IV therapy may be used to correct electrolyte imbalances, to deliver medications or nutrition, for blood transfusion or as fluid replacement to correct dehydration. IV therapy can also be used for chemotherapy of cancer patients. Fluid-transferring devices such as syringes may also be attached to a hub that connects a cannula for the addition or removal of fluid to/from a variety of bodily cavities, organs or vessels. For instance, the hub may be part of an entity providing a catheter to drain urine from the bladder or kidney, to remove fluid from an abscess, to extract liquid from joints or cysts, or to administer breathing gases through a tracheal tube. A typical endotracheal tube includes a cuff inflation tube with a hub for attachment of a syringe to enable inflation to seal the trachea and bronchial tree against air leakage and aspiration of fluids. A tracheostomy tube or urinary tract catheter might also use a cuff system with a hub for connection of a syringe or other device to inject fluid to inflate a cup or balloon that holds it in place. However fluid injections using a syringe connected to a needle are one of the most common health care procedures in the world.

When transferring fluids to/from a subject, the hub with its needle, catheter or other cannula inserted in the patient is often left in-situ while the fluid-transferring device may be removed and replaced, e.g. to empty/re-fill a syringe or to change over the IV therapy. Where two medical devices that carry small fluid volumes must be connected, a standard Luer fitting is the most common means of achieving a leak-free junction. One type of Luer fitting, commonly called a “Luer lock/lok”, uses an internally threaded collar surrounding a “Luer slip” friction fit (see below) tapered male tip of a syringe or the like. The projecting tip can be inserted into a corresponding female hub with an external thread and the collar screwed down to lock the connection. Such Luer lock fittings have the advantage of providing a secure connection that can not easily come loose, but two hands are needed to hold the hub while screwing the device in/out. A more rapid form of attachment may be preferred in some circumstances, for example in an emergency situation. Another type of Luer fitting, commonly called a “Luer slip”, simply uses a friction fit between a female hub and corresponding tapered male tip of a device without a threaded collar. A standard friction fit is achieved by a 6% taper. A Luer slip attachment is common for infusing less viscous fluids, such as vaccinations, and transferring fluids where high pressures are not involved, for example when drawing blood.

A problem observed with both Luer lock and Luer slip connections is the risk of injury when detaching the fluid-transferring device from a hub that is still connected to a patient. While a medical practitioner might take care to hold the hub and avoid injury when unscrewing a Luer lock connection, there is a temptation with a Luer slip connection to try to pull the device from the hub e.g. with one hand. However this can easily result in the hub being tugged away from the body and causing tissue damage. Often the device may not be pulled in a straight line with the cannula connected to the hub, but rotated, and this can twist the components. The tape used to hold the hub e.g. IV port in position is often loosened from the skin and its cannula e.g. needle may even be accidentally extracted. When emptying fluid from a body cavity, for example, keeping the needle hub still when detaching the syringe can be essential to avoid diffuse cutting inside the cavity or damage of the cavity wall. In addition there is a risk of unacknowledged contamination of both the hub and the Luer tip (not only the user) when holding the very small hub with the thumb and index fingers while pulling away the male tip, the tip sliding past the user's fingers as it is released.

Moreover tugging with a single hand does not usually apply enough force even to pull the device out of a friction fitting (such as a Luer slip) and, depending on the force used when connecting the Luer slip tip to the hub, the practitioner usually needs to hold or push the hub while also pulling the device so that it becomes detached. Typically the device will be rotated simultaneously while pulling away from the hub. This jerking can result in unwanted extraction of the needle or other component connected to the hub. The connection will often be pressurised by fluid. For example, a cuff connected to a tracheostomy tube, endotracheal tube or urinary catheter often has a tight connection of the male Luer tip with two-handed operation being required to loosen the connection while the sprung piston in the female Luer hub blocks the outflow of fluid (air or liquid) from the cuff.

Ease of disconnection can be a problem not only when detaching a device from a hub connected to a patient but also when it is desired to fill/empty a device such as a syringe via a fluid hub in a quick and convenient manner. For example, when filling a syringe using a needle inserted in a vial, each time that the syringe is removed it requires two hands to firmly grasp the needle hub and the syringe to separate them while the needle remains in the vial. As mentioned above, there is again a risk of contamination as the user grasps the hub and the tip comes into contact with the fingers holding the hub.

Another situation where a user might come into contact with a needle hub is when using a blood collection tube. The blood tubes are evacuated plastic or glass containers sealed with an elastomeric septum that is piercable by a double-ended needle to draw venous blood. Due to the piercing force and pressure differential, a secure connection to the needle assembly is required and therefore a threaded Luer lock connection is normally used rather than a Luer slip. U.S. Pat. No. 5,201,716 proposes an alternative blood specimen collection system that does not require the needle assembly to be grasped and twisted during disconnection. In this system a needle assembly is mounted with an interference fit rather than a threaded connection. A pivotally mounted lever assembly is spring-biased to hold the needle assembly in position, i.e. to provide an additional level of security over the friction fit. If the lever is actuated against its spring bias then there is only an interference fit holding the needle assembly in place. The lever can be pivoted to simultaneously release the spring bias and to apply a forward ejection force to the needle assembly.

In any situation where one hand is holding a needle hub while pulling a device away there is a risk of needlestick injury and contamination. Needle caps frequently being mislaid or forgotten can exacerbate this. This also applies when separating a needle or other contaminated component from a syringe or similar device for disposal purposes, with many needlestick injuries occurring when trying to remove sharps to throw into a bin. Usually the person handling a syringe will try to cover a contaminated needle with a cap after use, before grasping the hub to separate the needle from the syringe barrel for disposal. However, when mounting a needle cap onto the contaminated needle a person will use the large muscle groups in the arms and shoulders that work less precisely and, combined with poor depth of vision, this often results in a needlestick injury to the fingers holding the needle cap. It would be better if a needle hub could be safely released without needing to cap the needle or handle the connection.

The present invention seeks to address or mitigate the problems outlined above.

According to a first aspect of the present invention there is provided a fluid transfer device comprising: a fluid chamber in communication with a male connector tip, the male tip being tapered to form a friction fit when inserted in a corresponding female hub; a disconnecting member moveable relative to the male connector tip between a first position proximal to the fluid chamber and a second position spaced from the first position towards a distal end of the male connector tip; and means for gripping a hub when connected to the tip in use.

It will be understood that such a fluid transfer device provides an alternative solution to a conventional Luer Lock connection. Instead of a threaded connection in addition to the “Luer slip” friction fitting, the device provides gripping means to hold the hub in a locked position. The gripping means can provide a safety back-up to the friction fit and additional peace of mind that the connection will not fail during use. In at least some scenarios it may be preferable to provide additional means that can ensure a reliable and unfaltering connection in case the friction fit between the tapered tip of the fluid transfer device and a corresponding female hub can not be relied upon to provide a secure, fluid-tight connection. Preferably the additional gripping means can be activated and/or de-activated without requiring two-handed operation Such gripping means may be releasable by applying linear, rather than rotational, forces. Accordingly a user may be able to disconnect the device from a hub without twisting.

To be able to disconnect the device from a hub, a user may need to first release the gripping means. However it is preferable for this to be automatic and thus the disconnecting member may be arranged to release the gripping means when moving between the first and second positions. Accordingly the gripping means is preferably released by operation of the disconnecting member.

The gripping means may operate automatically when the tip is connected to a corresponding hub, for example comprising one or more latch members or snap-fit members. It will be understood that the gripping means is intended to take any suitable form other than an internally threaded collar. Advantageously the gripping means may be integrally formed with the fluid chamber so as to avoid additional parts. The gripping means may face inwardly or outwardly. The gripping means may be designed to engage with a custom hub or with a standard hub i.e. a standard Luer slip connector hub.

In one set of embodiments the gripping means takes the form of a pair of gripping fingers provided at a front end of the fluid chamber. The gripping fingers may extend forwards parallel to the tapered tip. The gripping fingers may be integrally moulded with the fluid chamber. In one example, the gripping fingers engage with an inside surface of the hub when it connected to the tip, e.g. having outwardly facing teeth. In another example, the gripping fingers engage with an outside surface of the hub e.g. having teeth arranged to grip onto the outside of the hub. Such gripping fingers may be designed to snap-fit onto the hub when it is connected to the fluid transfer device. The hub may be provided with a rim or other surface to facilitate engagement of the fingers. In another example, the gripping fingers may take the form of a pair of snap-fit fingers, or other gripping means, that positively engage i.e. grip a hub when it is connected to the tip. The hub may be provided with a ledge or other surface for the snap-fit fingers to grip. Such snap-fit fingers may be arranged to grip onto an outer surface of the hub or inside the hub.

In another set of embodiments the device may be arranged to be gripped by means provided on the female hub. Again, such gripping means may preferably be released by operation of the disconnecting member.

In a preferred set of embodiments the disconnecting member comprises a lever member pivotally connected to the device with one end, such as a front surface, moveable between the first and second positions relative to the male connector tip. In embodiments where the gripping means takes the form of a pair of gripping fingers, the lever may pivot to push the fingers inwardly or outwardly to release their grip on the hub, e.g. while the hub is pushed along the tip. In embodiments where the gripping means takes the form of a pair of snap-fit fingers, two lever members pivotally mounted at the front of the fluid chamber may pivot so as to cause the fingers to flex outwards so that the hub is released from their grip.

The disconnecting member may be part of a lever mechanism. In fact the device may not even comprise a fluid chamber as such a lever could interact directly with the male tip that provides fluid transfer, for example if the fluid chamber is removable or if fluid is transferred directly to the tip. The fluid transfer device may have a tapered tip but no fluid chamber of its own to provide for fluid storage, for example a fluid transferring connection for a hose or tubing rather than for a syringe. Furthermore, while the present invention has been described so far in the context of a fluid transfer device comprising a “male” tapered tip, it will be appreciated that a friction fit between a device and a corresponding hub may equally be achieved by reversing the male and female parts of the connection. Accordingly, in each embodiment of the invention disclosed above the male tip could be replaced with a tapered female part that forms a friction fit when a corresponding male part provided by a hub is inserted therein instead. The disconnecting member would still act to release the friction fit, this time moving along the female part to push away the male part inserted therein. Although such embodiments depart from the standard design of a Luer slip connection for fluid transfer devices such as syringes, it is envisaged that a new standard could be implemented with the male and female parts reversed as outlined here.

A gripping means is described above as providing a safety back-up to the friction fit of a standard Luer slip connection. The reliability of such an additional gripping means may depend on how tightly it is able to grip a hub and hold it in a locked position when also connected to the fluid transfer tip by a friction fitting. The Applicant has recognised that an improvement can be provided in the form of a “double lock”, in other words, using an additional gripping means to lock the hub on the tip and then locking that grip. Furthermore the Applicant has recognised that the disconnecting member can conveniently assist in holding the hub in the locked position i.e. providing the “double lock”. This can be arranged to occur automatically by providing the disconnecting member with a resilient bias. In a preferred set of embodiments the disconnecting member is provided with a resilient bias that causes it to engage the gripping means and thereby assist in holding the hub in the locked position.

This is considered novel and inventive in its own right, and thus according to a second aspect of the present invention there is provided a fluid transfer device comprising: a fluid transfer tip, the fluid transfer tip comprising a tapered friction fitting for a corresponding hub; an additional means for gripping a hub in a locked position when connected to the fluid transfer tip by the friction fitting; and a disconnecting member provided with a resilient bias that causes it to engage the gripping means and thereby assist in holding the hub in the locked position, wherein the disconnecting member is moveable relative to the fluid transfer tip and against the resilient bias so as to release engagement of the gripping means, to move the hub out of the locked position provided by the gripping means, and to release the hub from the friction fitting.

It will be appreciated that, similar to the first aspect described above, the device provides additional means for gripping a hub in a locked position as an alternative to a Luer lock screw connection. This second aspect of the invention further provides the security of assisting the gripping means in holding the hub in the locked position, i.e. a “double lock”. The disconnecting member advantageously uses its resilient bias to automatically assist the gripping means. A hub is disconnected from the tip in a three-stage process. First the disconnecting member is moved against its resilient bias so as to release engagement of the gripping means. Then the disconnecting member acts to move the hub out of the locked position provided by the gripping means, e.g. movement of the disconnecting member overcomes the locking grip. Finally, the disconnecting member releases the hub from the friction fitting, e.g. by pushing the hub out of the tight fit provided by the taper. The disconnection process may be provided by a single, smooth movement of the disconnecting member.

In one set of embodiments the disconnecting member may be pivotally mounted to the device. Preferably the disconnecting member comprises at least one lever member pivotally mounted to the device. An advantage of using a lever member to disconnect the tip from a corresponding hub is that it can amplify an input force to provide a greater output force, i.e. providing leverage to push a hub away from the tip. The mechanical advantage of a lever member can increase the force applied so that the device can be released without necessarily holding the hub, thereby enabling single-handed operation. Furthermore, a lever member can be ideally suited to engage the gripping means when pivoted into one position and to move the hub out of the locked position provided by the gripping means as it pivots into another position, with further pivotal movement of the lever member also acting to release the hub from the friction fitting.

In order for the lever member to transfer force efficiently, it is preferable for it to be relatively stiff. However it may also be desirable to mould the device, or at least the lever member, from plastics materials so as to provide a cheap, sterile and disposable product for single use in a medical setting. The lever member may be stiffened by forming it as a three-dimensional shell. Preferably the lever member comprises a front surface that is substantially transverse to the axis of the tip and one or more side surfaces that extend in a direction substantially parallel to the axis of the tip. Preferably the surfaces form a shroud extending back from the front surface and away from the fluid transfer tip. The three-dimensional extent of the member can help to ensure that it is stiff even if formed of a plastics material, as is preferred in various embodiments.

In a set of embodiments the lever member has an at least partially cylindrical form with the side surface(s) extending substantially parallel to the axis of the fluid transfer tip being cylindrical side surfaces. The side surface(s) do not need to fully surround the axis of the fluid transfer tip. But in at least one set of embodiments the front surface of the lever member is connected to one or more side surfaces that surround the fluid transfer tip. This can stiffen the lever member so that the front surface preferably does not flex when pushed against a hub but instead transmits its kinetic energy to move the hub away.

Alternatively, or in addition, the front and side surface(s) of the lever member are preferably integrally formed. For example, at least these parts of the lever member may be formed as a single plastics moulding. Alternatively, or in addition, it is preferable that the front surface at least partially surrounds the fluid transfer tip. The front surface may entirely surround the fluid transfer tip, for example with the tip protruding through an aperture in the front surface. This can make the lever member more compact and/or make the front surface more effective in pushing against a hub mounted on the fluid transfer tip with a friction fit.

A further advantage of using a lever member to disconnect the hub is that the shape, in particular the curvature, of the front surface can be designed to control the leverage that is achieved. In one set of embodiments the front surface is curved such that initial movement of the lever member against the resilient bias moves the front surface substantially transverse to the fluid transfer tip to release engagement of the gripping means and further movement of the lever member against the resilient bias moves the front surface along the fluid transfer tip to move the hub out of the locked position provided by the gripping means and release the hub from the friction fitting. Accordingly the curvature of the front surface provides for two different movements that are matched to the different stages of disconnection.

While a lever member is a good example of a disconnecting member that can apply sufficient force to overcome the gripping means and release the hub from its friction fitting, a potential problem with pushing a hub away from a tip is that it may be forcibly disconnected. If the hub is carrying a needle or other sharp object then this could pose an injury risk. It is therefore preferable that the device further comprises a catch means arranged to catch the hub after it has been released from the friction fitting. Preferably further movement of the disconnecting member against the resilient bias causes the catch means to catch the hub. In this way the hub may be caught as it becomes disconnected but then controllably separated from the device. The catch means be may be subsequently released by resiliently biased movement of the disconnecting member, e.g. back to its resting state.

The additional gripping means may take any suitable form other than the internally threaded collar of a Luer lock connection. For example, it is mentioned above that suitable gripping means may include one or more latch members, snap-fit members or gripping fingers. The gripping means may be configured to grip a standard Luer slip hub, for example gripping onto the rim at the base of the hub. Alternatively, or in addition, the gripping means may be configured to grip a standard Luer lock hub but without a screw connection to the outer thread. For example, it is envisaged that gripping fingers could latch onto the outer thread of a Luer lock hub and be held in a locked position until the disconnecting member is moved against its resilient bias. This would allow for disconnection without having to twist the device relative to the hub.

The Applicant has recognised that an alternative hub design may be used to ensure a tight grip. The hub may comprise both a tapered surface to form a friction fit with a corresponding fluid transfer tip and an additional receiving means allowing a fluid transfer tip to grip the hub when the hub is connected thereto by the friction fit. The additional receiving means may comprise a flange or groove. For example, a female hub may comprise a tapered internal surface and the additional receiving means may comprise a flange or groove also provided on the internal surface. Preferably the fluid transfer tip is provided with a corresponding groove or flange, for example on an outer surface of a male fluid transfer tip that is to be inserted into a female hub, that engages with the receiving means to hold the two parts together.

Turning back to the fluid transfer device, in one set of embodiments the additional means for gripping a hub comprises a flange or groove provided on the fluid transfer tip. The flange or groove may engage with a corresponding groove or flange provided on a corresponding hub. The gripping means may be held in a locked position by the disconnecting member acting to reinforce this physical engagement. In a preferred embodiment the disconnecting member has a surface that is resiliently biased to at least partly surround the flange or groove. Where the disconnecting member comprises a lever member with a front surface that is substantially transverse to the axis of the tip and at least partially surrounds the tip, the tip may pass through an aperture in the front surface that has walls arranged substantially parallel to the tip so as surround the flange or groove. Preferably these walls apply pressure around the flange or groove when the lever member is resiliently biased into a position to assist locking of the hub. The aperture may be dimensioned so that its walls no longer hold the flange or groove in engagement with the hub once the lever member pivots against its resilient bias.

The aperture in the lever member, or any other surface of a disconnecting member, may vary in diameter so as to provide a tight fit surrounding the flange or groove only when the disconnecting member is resiliently biased. The disconnecting member can then automatically engage or release the gripping means depending on its position relative to the resilient bias. In one example the disconnecting member has a surface that is shaped to slide against a hub in the vicinity of the flange or groove.

There have already been described above some embodiments in which the gripping means takes the form of gripping fingers that can engage with a standard Luer slip hub or a novel hub design. In embodiments of the second aspect of the invention such gripping fingers are held in their locked position so as to ensure that a hub can not be accidentally released e.g. during a fluid transfer procedure. In one set of embodiments the additional means for gripping a hub comprises one or more fingers arranged to grip onto an outer or inner surface of the hub. For example, the fingers may be arranged to grip onto an outer surface of a rim on the hub. In another example, the fingers may be arranged to grip onto an outer thread on the hub. Preferably the disconnecting member has one or more supports aligned with respective fingers and the disconnecting member is resiliently biased to press the support(s) against the finger(s) in the locked position. The support(s) can therefore act to strengthen the gripping fingers and ensure that they do not flex when holding a hub in the locked position. It is preferable that the pressure provided by the support(s) is automatically released when the disconnecting member is moved against its resilient bias so as to release engagement of the gripping means. Accordingly the disconnecting member may be moveable against the resilient bias to release the support(s) and thereby release the fingers. The fingers may then be free to flex away as the hub is moved out of the locked position released from the friction fitting. However it may be preferable for the fingers to be positively moved out of gripping engagement so that they can not hinder release of the hub. Accordingly the disconnecting member may be further moveable against the resilient bias to push the fingers away from the hub.

In one example, the fingers may comprise one or more gripping teeth. Alternatively, or in addition, the fingers may comprise at least one thread arranged to engage with a thread on the hub. Alternatively, or in addition, the fingers may comprise a stepped gripping surface.

In one set of embodiments the additional means for gripping a hub comprises a latch mechanism at the base of the fluid transfer tip. For example, the latch mechanism may comprise one or more snap-fit members. It is preferable that the disconnecting member has a surface that is resiliently biased to bear on the latch mechanism and thereby assist in holding the hub in the locked position. The disconnecting member may have a surface that varies in diameter so as to bear on the latch mechanism only when the disconnecting member is moved by the resilient bias.

In addition to (or instead of) a surface or supports arranged to apply pressure to a gripping means and thereby assist in holding the hub in the locked position, the disconnecting member may ensure the integrity of such “double locking” by positively engaging with the hub in another way. In one set of embodiments the disconnecting member comprises at least one latch member arranged to engage with the hub and assist in holding the hub in the locked position. The latch member may be engaged and/or disengaged independently of movement of the disconnecting member. However it is preferable for the latch member to be engaged and/or disengaged automatically. Preferably it is movement of the disconnecting member against the resilient bias that disengages the at least one latch member from the hub. The latch member may engage with an inside or outside surface of the hub.

The disconnecting member may be moveable between two or more different positions relative to the fluid transfer tip. In one example, the disconnecting member is provided with a second resilient bias acting to oppose the first resilient bias so that an intermediate position can be achieved.

In some embodiments it may be helpful for a user to move the disconnecting member against the resilient bias at the same time as mounting a hub to the fluid transfer tip. This may help to ensure disengagement of the gripping means before the hub is fully connected, otherwise the disconnection member might interfere with mounting the hub. However it is preferable for this to be achieved automatically so that a user merely has to push a hub onto the tip. In one set of embodiments the disconnecting member comprises a surface that is arranged to be acted upon by a hub as it is connected to the fluid transfer tip such that the disconnecting member is temporarily moved against the resilient bias to accommodate the hub.

In a set of embodiments the disconnecting member comprises a slot and the fluid transfer tip passes through the slot, the slot providing space around the tip to accommodate the hub when the disconnecting member is temporarily moved against the resilient bias. The surface arranged to be acted upon by a hub may be provided by such a slot.

In a set of embodiments the surface arranged to be acted upon by a hub is provided by a nose extending substantially parallel to the fluid transfer tip. For example, the disconnecting member may comprise a nose extending substantially parallel to the fluid transfer tip and having an angled forward surface that is arranged to be acted upon by a hub as it is connected to the fluid transfer tip.

In either set of embodiments, the disconnecting member may be moved by the resilient bias to engage the gripping means after the hub has been accommodated and connected to the tip.

It may be desirable to disable the resilient bias when the device is not in use, for example to make it compact for storage and/or transport. A generally applicable feature is for the device to comprise means to lock the disconnecting member against the resilient bias.

Embodiments of the second aspect of the invention described above benefit from the security of “double locking” i.e. a gripping connection (in addition to the friction fitting) that is then locked by automatic engagement of the disconnecting member. However the Applicant has recognised that a double lock may not be necessary if the gripping connection is strong enough on its own, for example a screw fit. However it is still advantageous to take advantage of a disconnection mechanism using a lever member for force amplification.

Thus when viewed from a third aspect of the present invention there is provided a fluid transfer device comprising: a fluid transfer tip, the fluid transfer tip comprising a tapered friction fitting for a corresponding hub; a lever member pivotally mounted to move relative the fluid transfer tip; and a screw thread mounted on the lever member to enable a hub to be connected to the tip by a screw fit in addition to the friction fitting; wherein the lever member is resiliently biased so that the screw thread is positioned to form a screw fit with the hub; and wherein the hub can be disconnected by pivoting the lever member against the resilient bias to release the screw fit with the hub and to subsequently release the hub from the friction fitting.

Such a device provides a novel mechanism for locking with a screw fit and automatic release using a lever. The mechanism may be combined with one or more additional gripping means as already described above. For example, the device may further comprise fingers that have a thread to additionally grip the hub when the lever member is resiliently biased to form a screw fit. Such fingers may be arranged to disengage from the hub by the lever member pivoting against the resilient bias.

While the lever member may take many different forms, preferably the lever member comprises a front surface that is substantially transverse to the axis of the tip and the front surface is arranged to move along the tip when the lever member is pivoted against the resilient bias. Any of the other features of a lever member already described above may equally be applied to this aspect of the invention.

The Applicant has further recognised that a resilient bias may not be an essential feature to at least some further aspects of the invention. Thus when viewed from a fourth aspect of the present invention there is provided a fluid transfer device comprising: a fluid transfer tip, the fluid transfer tip comprising a tapered friction fitting for a corresponding hub; an additional means for gripping a hub in a locked position when connected to the fluid transfer tip by the friction fitting; and a disconnecting member comprising an actuator portion and operable to move the actuator portion between at least two different positions, wherein in a first position the actuator portion engages the gripping means and thereby assists in holding the hub in the locked position and in a second position the actuator portion does not engage the gripping means but acts to move the hub out of the locked position and release the hub from the friction fitting.

It is preferable that the disconnecting member is manually operable. A resilient bias may optionally be used to assist manual operation. In one example, the disconnecting member is provided with a resilient bias that moves the actuator portion into the first position. In one example, the disconnecting member is operable to move the actuator portion into a third position, between the first and second positions, where the actuator portion releases the gripping means and allows a hub to be connected to the fluid transfer tip by the friction fitting.

The disconnecting member may act in a similar manner to a lever member as already described. In one example the actuator portion extends in a plane substantially transverse to the axis of the fluid transfer tip. The actuator portion is preferably arranged to move along the tip when the disconnecting member is moved between the different positions. In one set of embodiments the disconnecting member comprises one of more lever members.

It will be appreciated that the various aspects of the present invention are not limited to a male transfer tip. In one set of embodiments the fluid transfer tip comprises a male connector tip that is tapered to form a friction fit when inserted in a corresponding female hub. However, in another set of embodiments the fluid transfer tip comprises a tapered female part that forms a friction fit when a corresponding male part provided by the hub is inserted therein.

Any suitable hub may be connected to the fluid transfer device, at least by the friction fitting. The hub may be a standard Luer slip hub, for example a hub comprising a tapered internal surface and an outer rim at its base. Or the hub may be a standard Luer lock hub, for example a hub comprising a tapered internal surface and an outer thread at its base. In some preferred embodiments there is provided a novel hub comprising a tapered internal surface and an outer rim or thread spaced from its base by a skirt portion.

The present invention extends to a hub for directing fluid from a fluid transfer device, the hub comprising a tapered internal surface to form a friction fit with a corresponding fluid transfer tip and an outer rim or thread to enable it to be positively engaged when connected to a fluid transfer tip by the friction fit, wherein the outer rim or thread is spaced from a base of the hub by a skirt portion. The skirt portion is useful as it can interact with the disconnecting member. This can be particularly beneficial when the friction fitting is supplemented by an additional gripping means. Thus the hub may further comprise an additional means for gripping a fluid transfer tip when the hub is connected thereto by the friction fit.

The present invention also extends to a hub for directing fluid from a fluid transfer device, the hub comprising a tapered surface to form a friction fit with a corresponding fluid transfer tip, a threaded collar or rim external to the tapered surface, and an additional means for gripping a fluid transfer tip when the hub is connected thereto by the friction fit. To ensure compatibility with at least some of the embodiments of a fluid transfer device described above, the additional means for gripping a tip may comprise a flange or groove provided on the tapered surface. As already mentioned, the threaded collar or rim may be spaced from a base of the hub by a skirt portion.

In at least some embodiments it is preferable that the skirt portion is flexible. This may be achieved by using the flexibility of the hub material and/or design of the hub, for example splitting the skirt portion into cut segments. If the skirt portion is split into separate, downwardly extending segments then it may be desirable for the segments to be arranged to provide flexibility but without making the skirt overly prone to splitting open. In one example, the skirt portion may be split into an odd number of equal segments, e.g. three, five, seven, etc. so that no two segments are ever diametrically aligned. This means that there is always a skirt segment on at least one side of a given diameter which can be engaged by the disconnecting member to assist in holding the hub locked onto the tip.

The fluid transfer device may comprise any type of device used to transfer fluid—liquid and/or gas—either to or from a fluid receptacle. The fluid receptacle may be inanimate or it may be part of a living subject, for example a bodily cavity, organ or vessel, such as a vein or artery. Although the present invention may find a wide range of uses, preferably the fluid transfer device is a medical device. The fluid transfer device may comprise one or more devices such as a syringe, pre-filled syringe, IV delivery device e.g. “drip”, transfusion device, fluid pump, stopcock, aspirator, suction device, container for a blood collection tube or hose. The device may be made to meet the relevant medical standard(s), for example ISO 7886 for sterile hypodermic syringes. In one set of embodiments hub comprises a cannula or hypodermic needle. In such embodiments the device may take the form of a syringe.

Some embodiments of the present invention will now be described, by way of example only, and with reference to the accompanying drawings, in which:

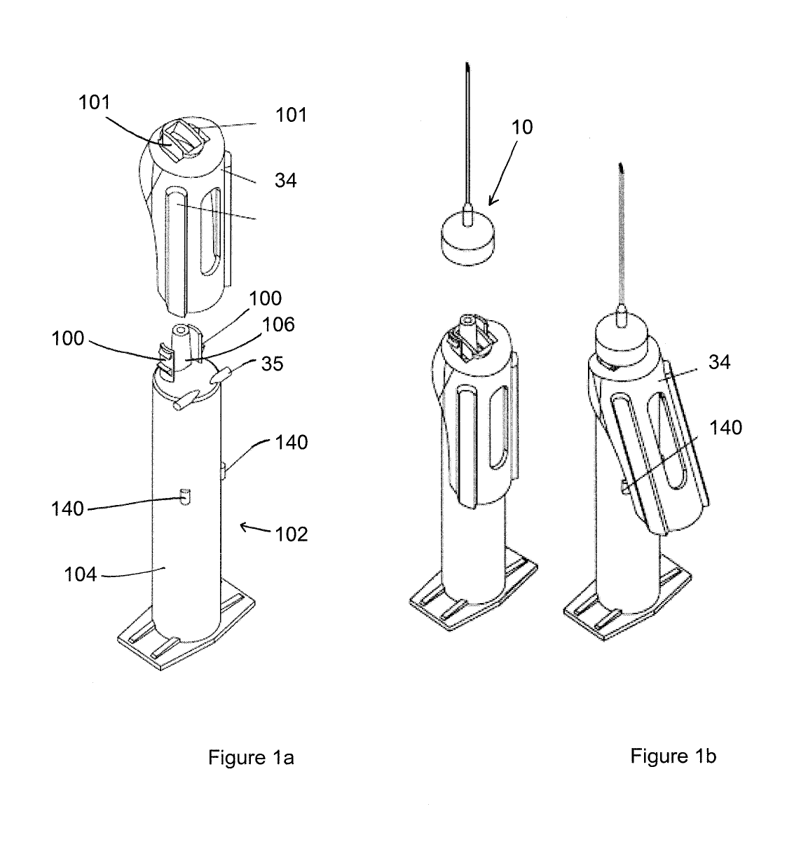

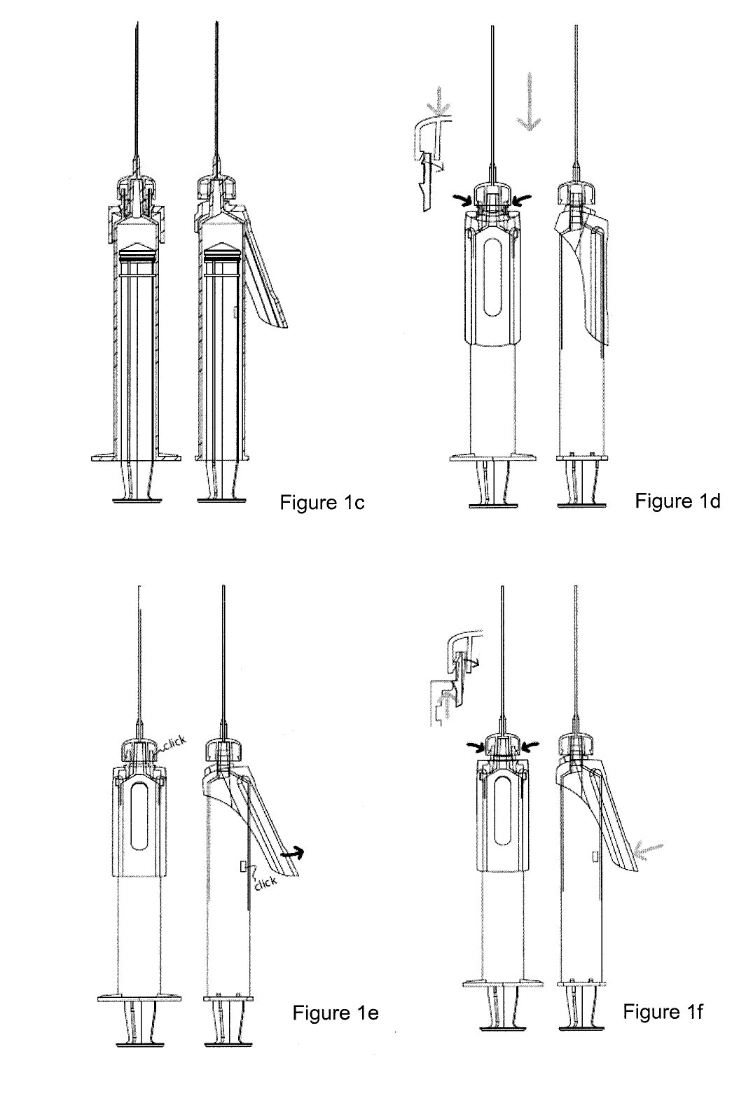

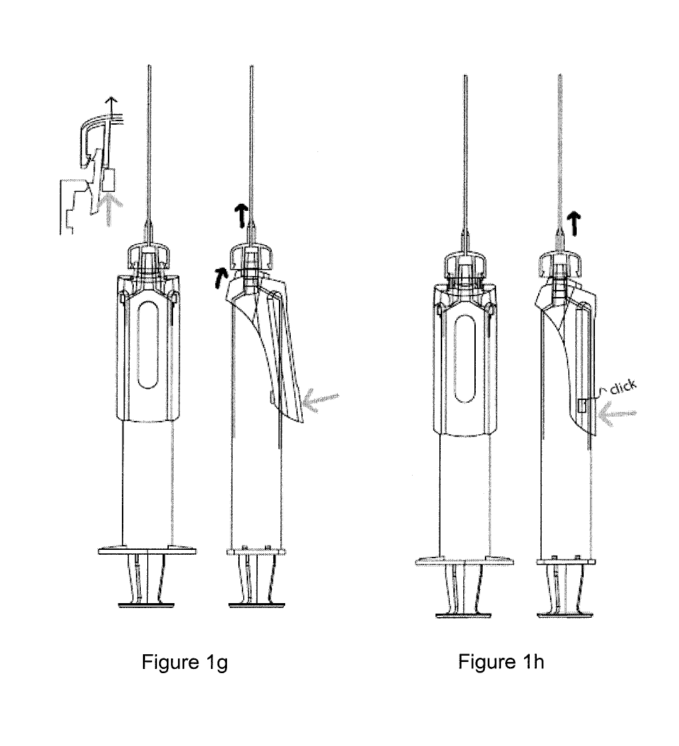

FIGS. 1a to 1h show a first embodiment of a disconnecting mechanism for a syringe showing connection to a first needle hub;

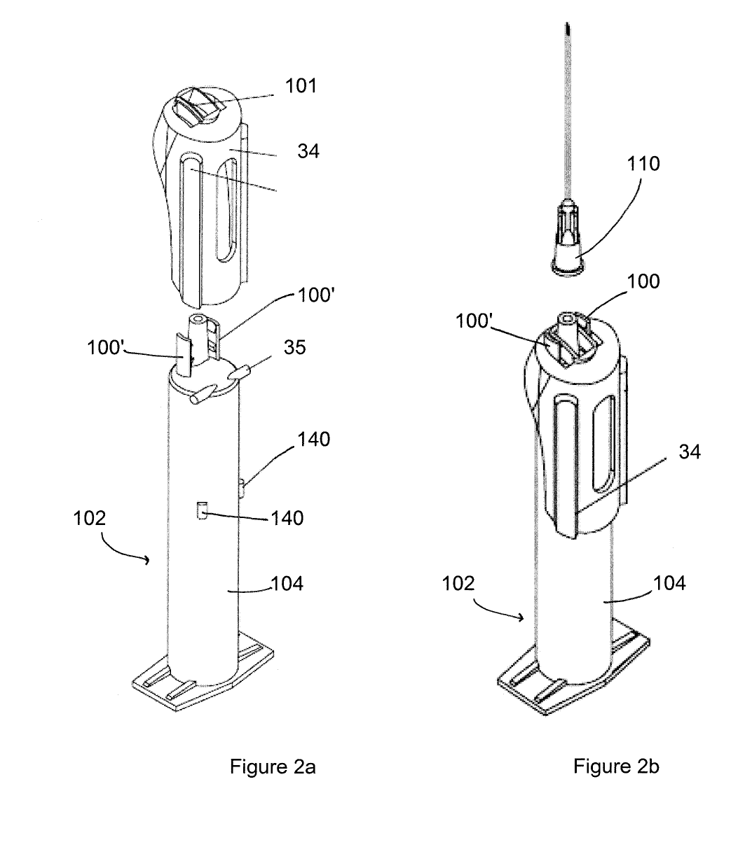

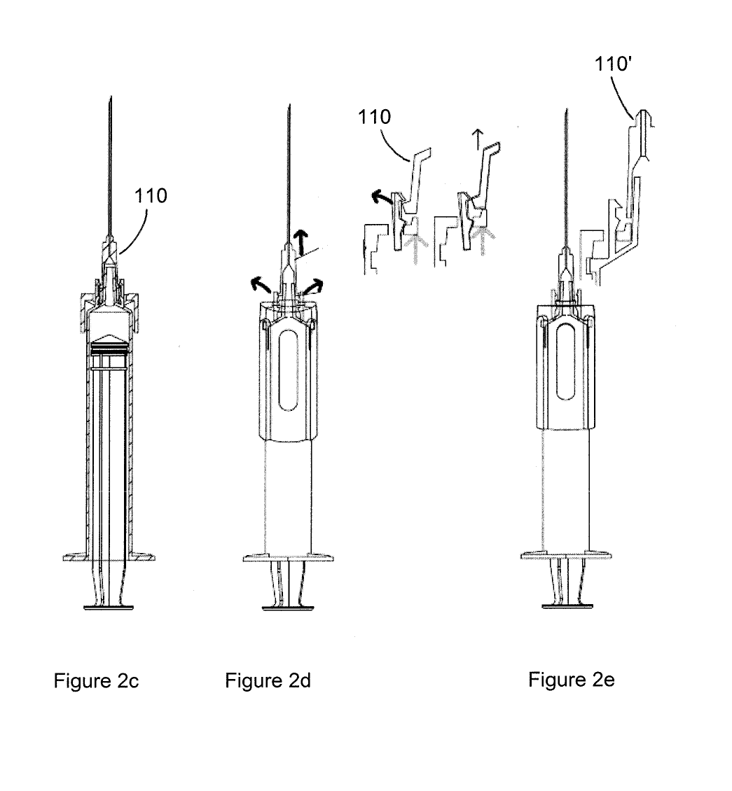

FIGS. 2a to 2e show a second embodiment of a disconnecting mechanism for a syringe showing connection to a second needle hub;

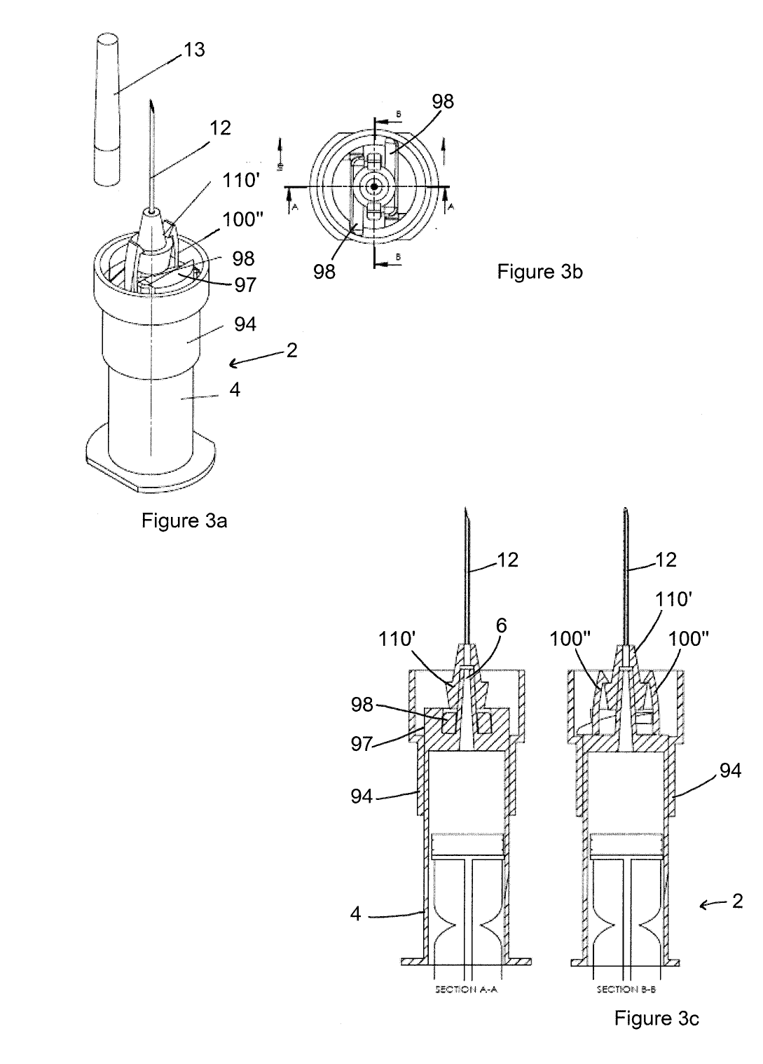

FIGS. 3a to 3c show a third embodiment of a disconnecting mechanism for a syringe showing connection to a third needle hub;

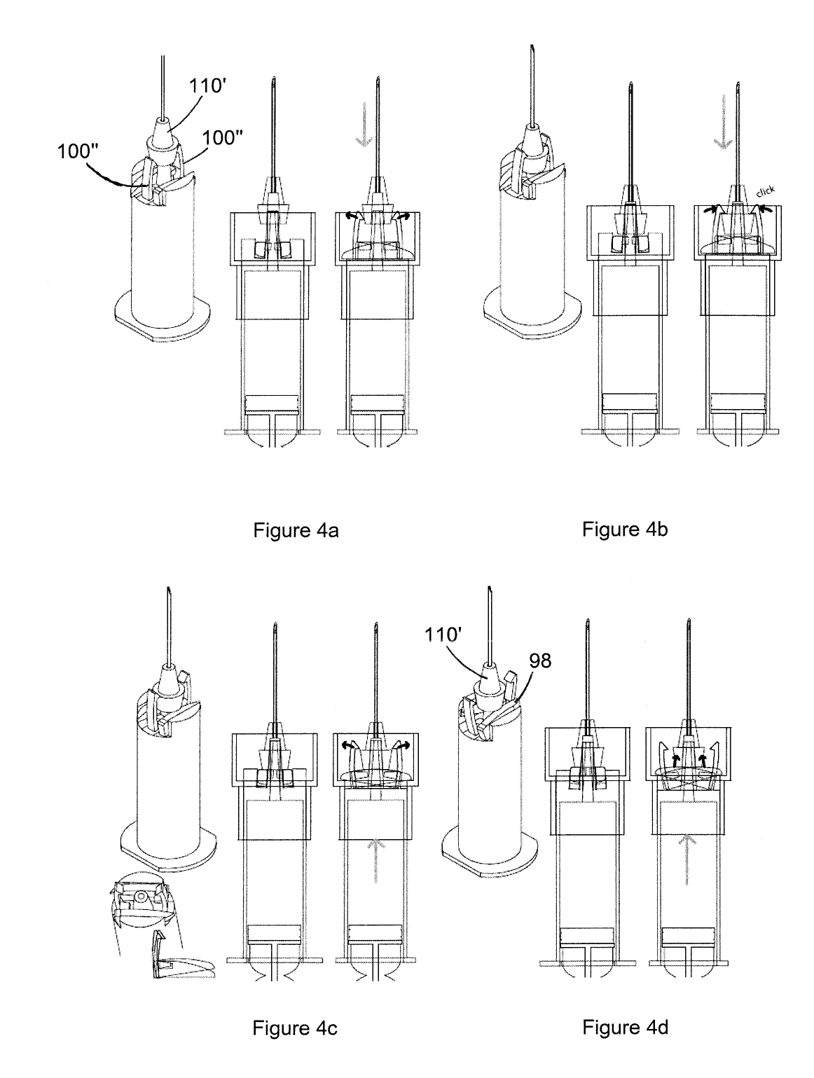

FIGS. 4a to 4d show operation of the embodiment of FIGS. 3a to 3c;

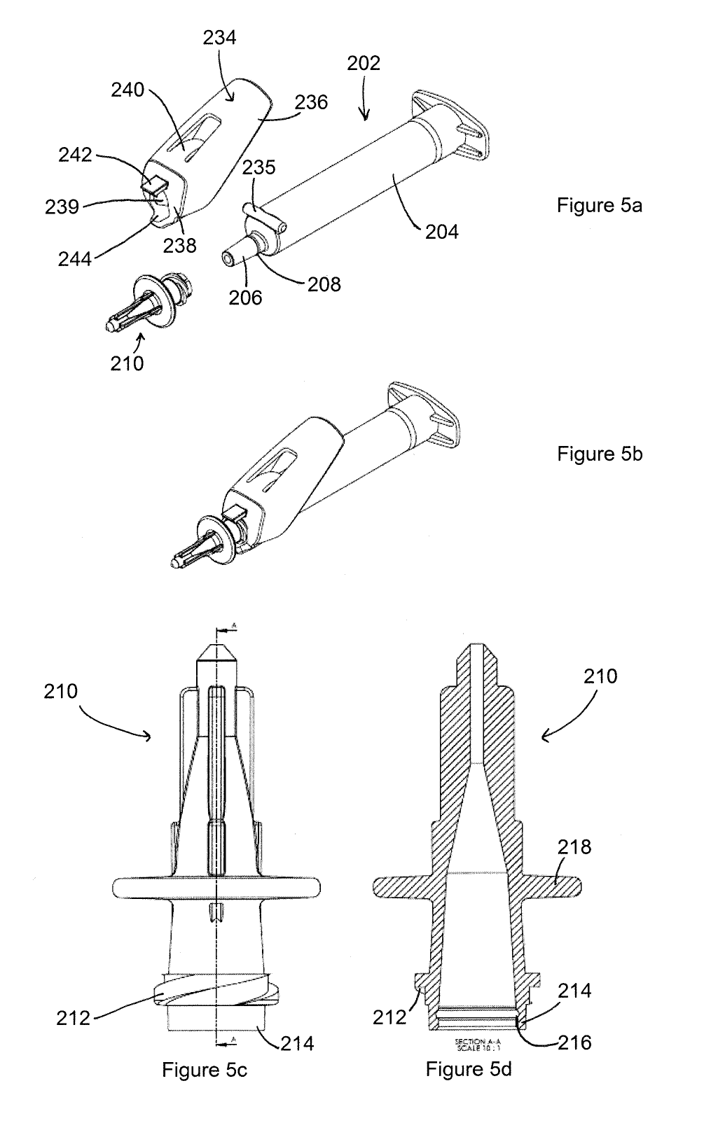

FIGS. 5a to 5d show a fourth embodiment of a disconnecting mechanism for a syringe showing connection to a fourth needle hub;

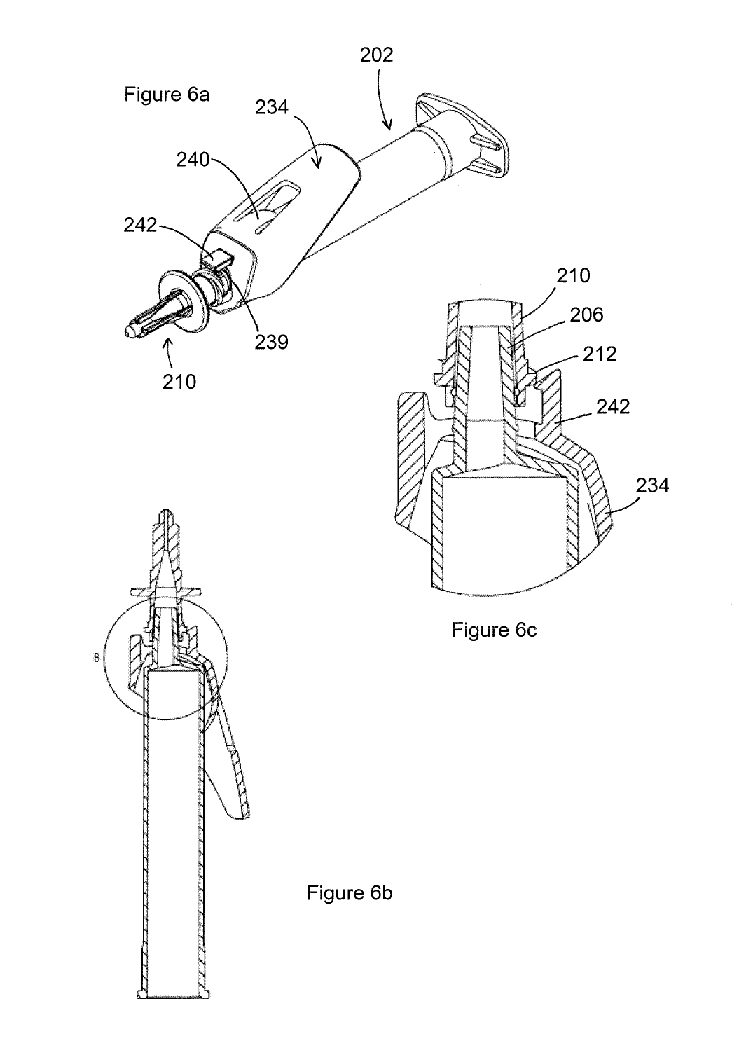

FIGS. 6a to 6c, 7a to 7e, 8a to 8e, 9a to 9e, 10a to 10e, 11a to 11e and 12a to 12e show various stages of operation of the fourth embodiment of a disconnecting mechanism;

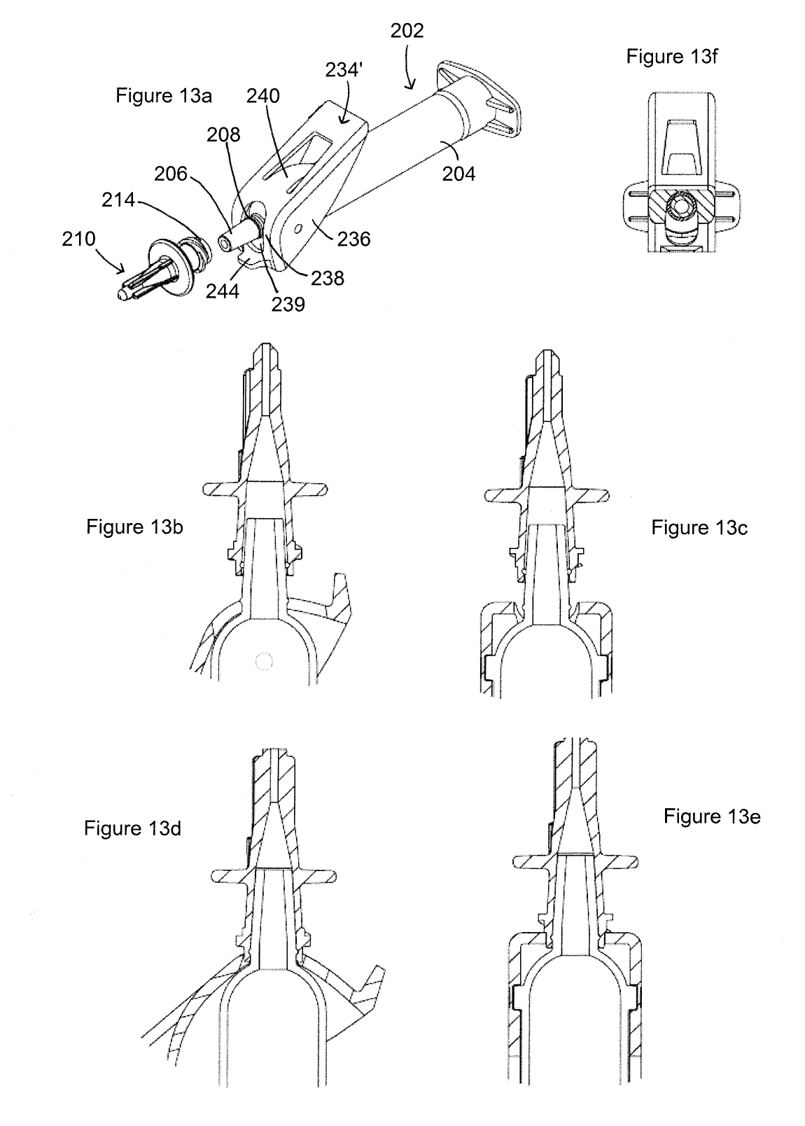

FIGS. 13a to 13e show a fifth embodiment of a disconnecting mechanism for a syringe showing connection to the fourth needle hub;

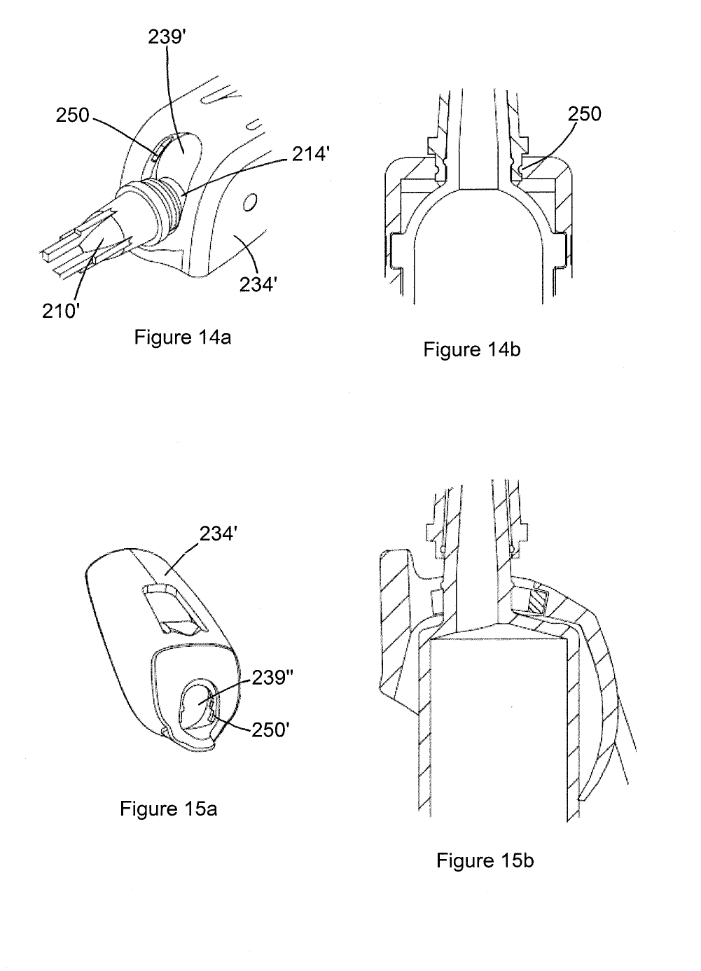

FIGS. 14 and 15 show some variants of the fifth embodiment;

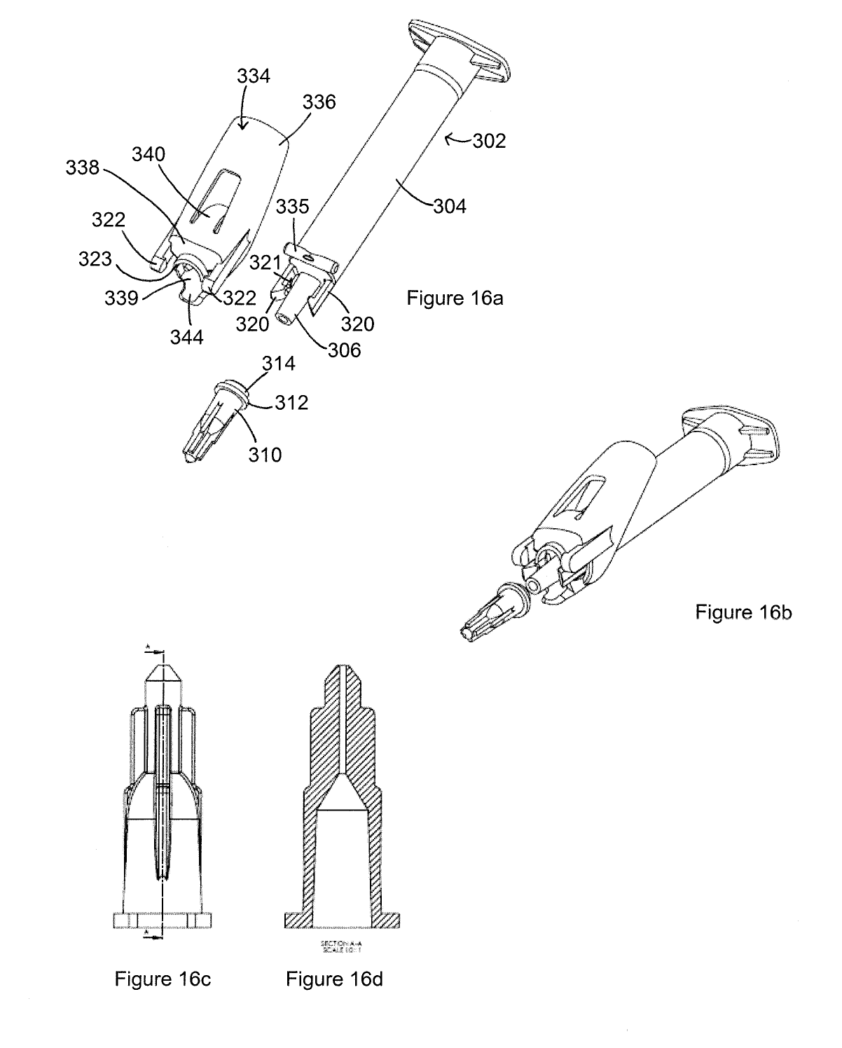

FIGS. 16a to 16d show a sixth embodiment of a disconnecting mechanism for a syringe showing connection to a fifth needle hub;

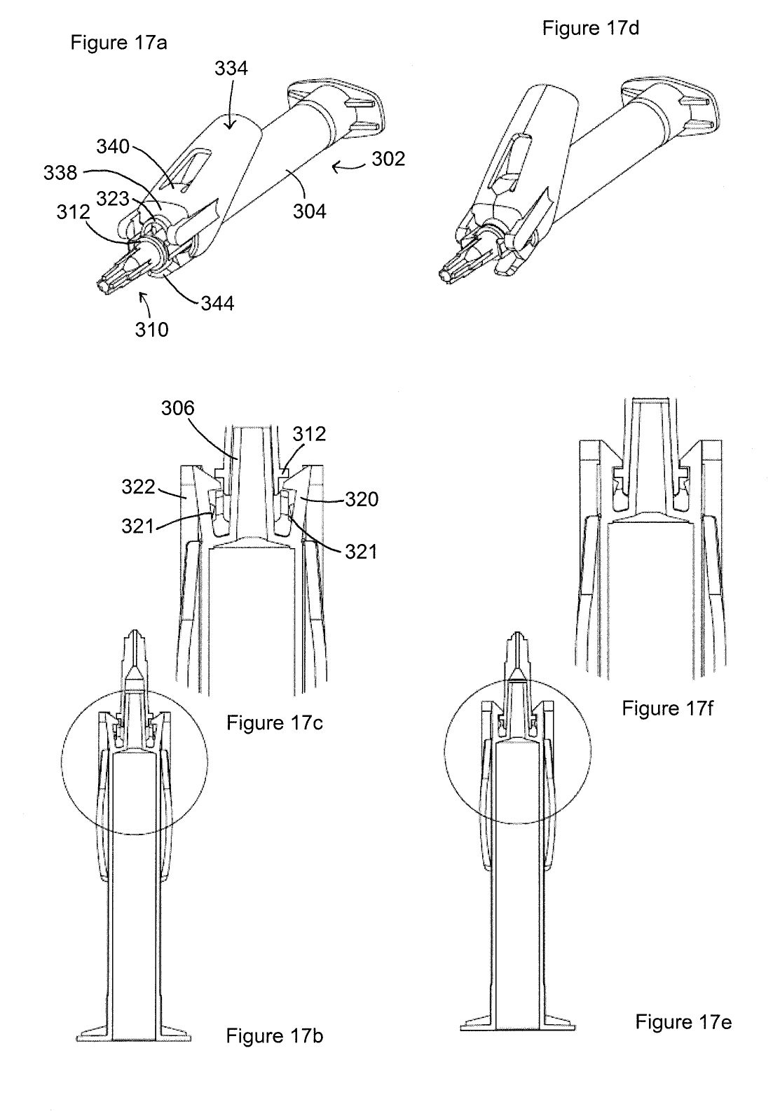

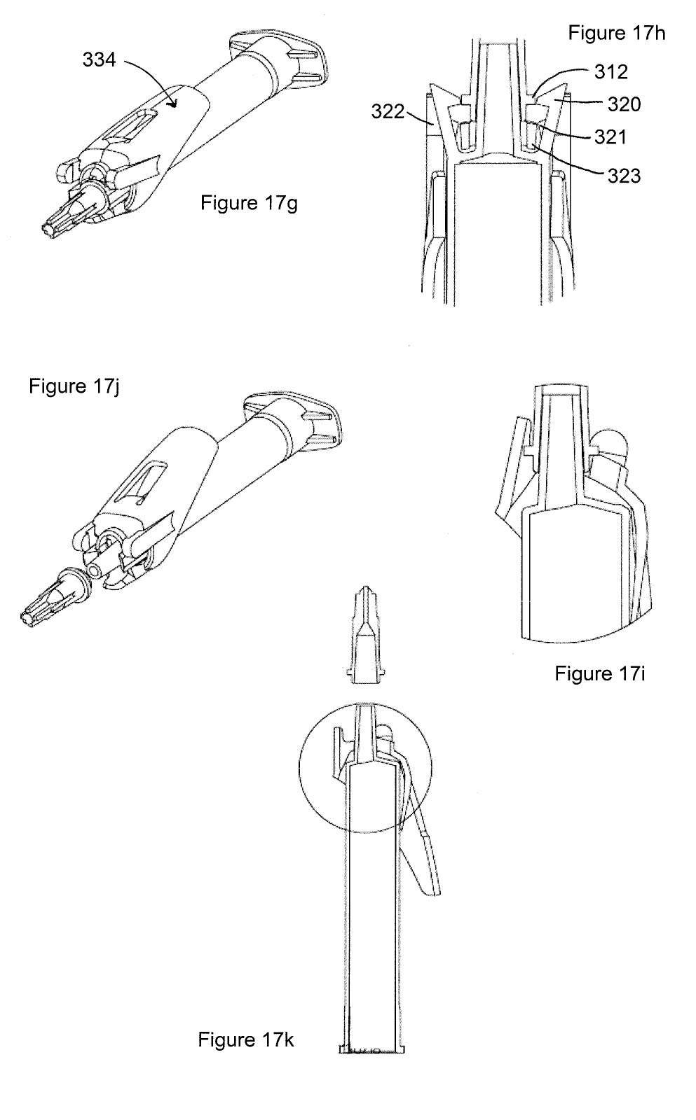

FIGS. 17a to 17i show various stages of operation of the sixth embodiment of a disconnecting mechanism;

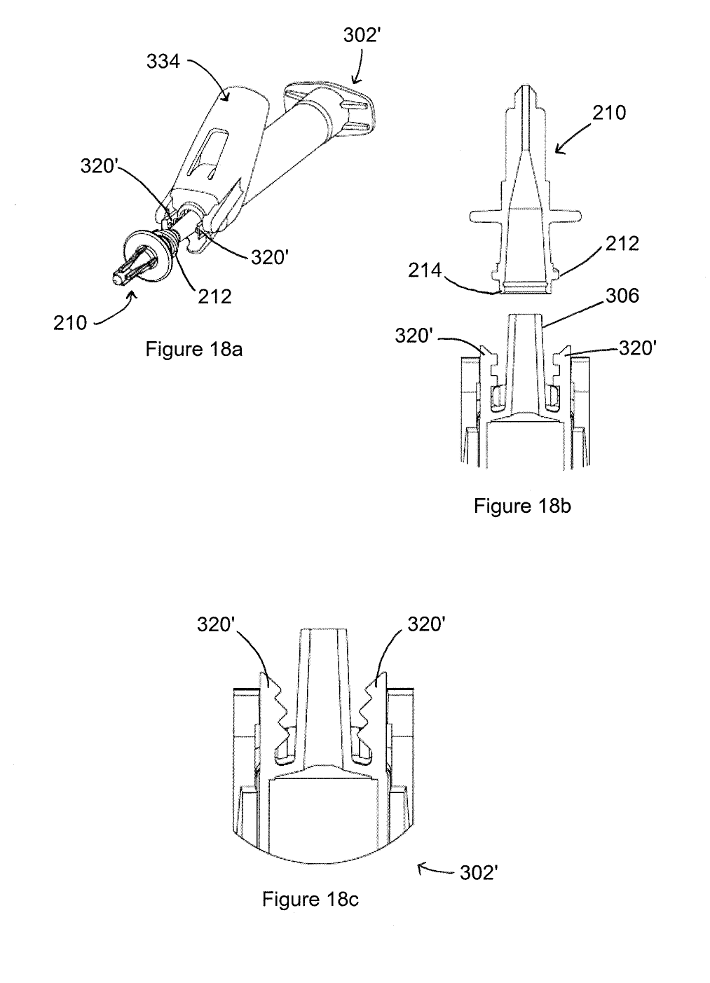

FIGS. 18a to 18c show some variants of the sixth embodiment;

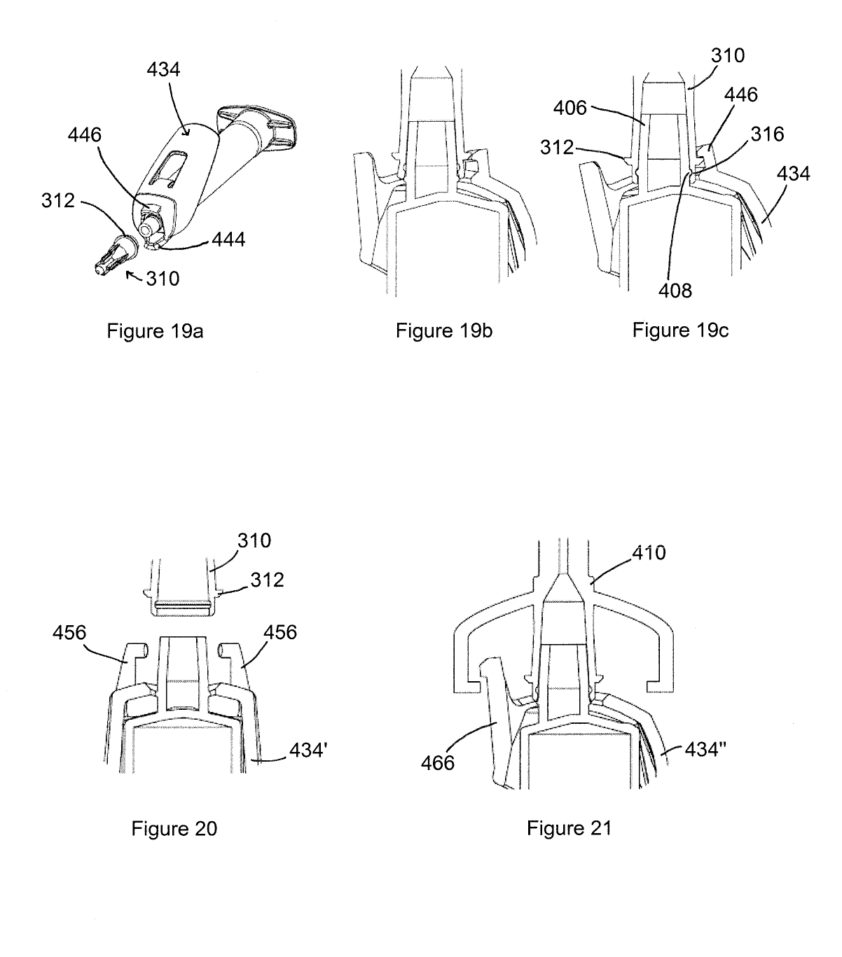

FIGS. 19a to 19c show a seventh embodiment of a disconnecting mechanism for a syringe showing connection to the fifth needle hub;

FIGS. 20 and 21 show some variants of the seventh embodiment;

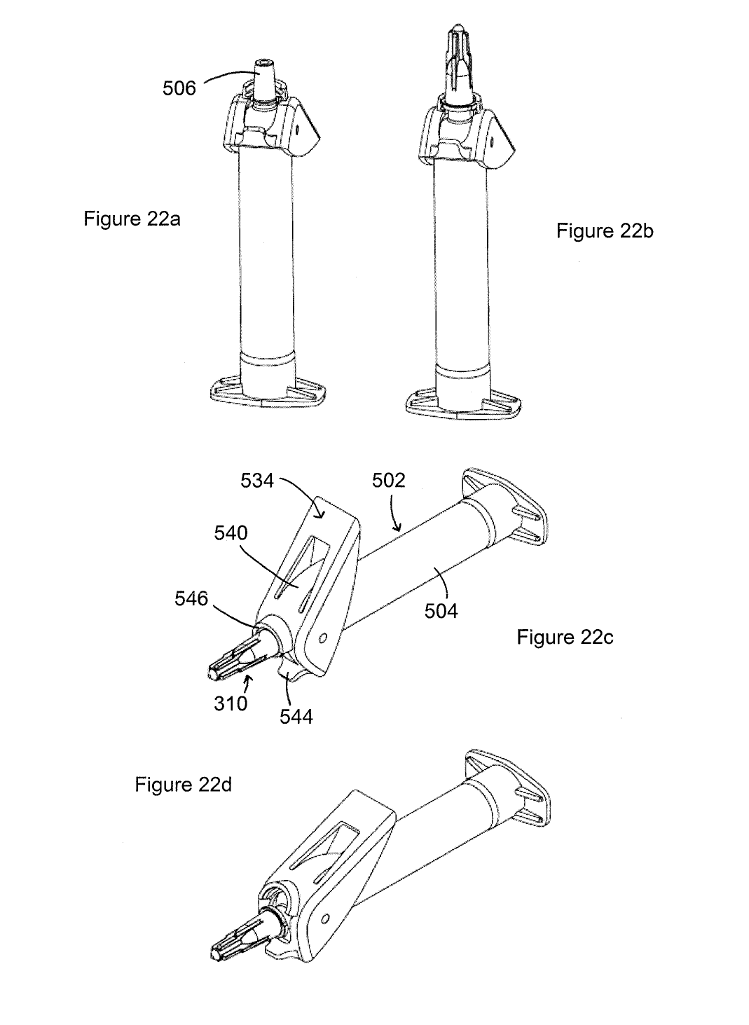

FIGS. 22a to 22d show an eighth embodiment of a disconnecting mechanism for a syringe showing connection to the fifth needle hub;

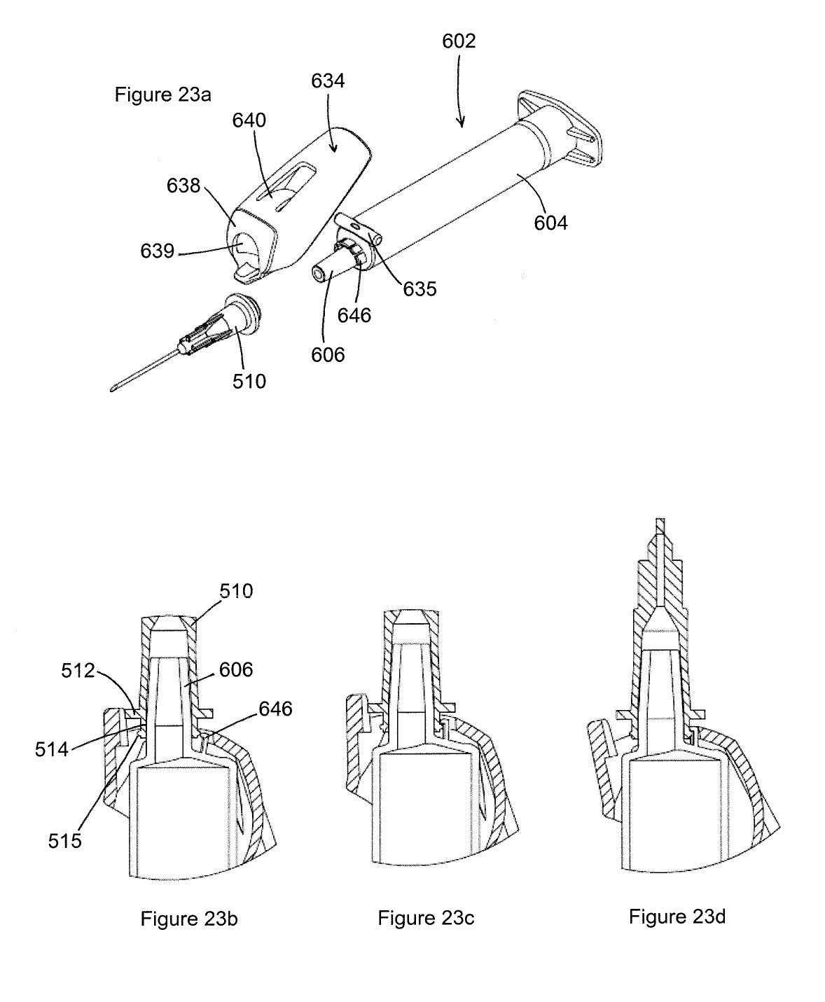

FIGS. 23a to 23d show a ninth embodiment of a disconnecting mechanism for a syringe showing connection to a sixth needle hub;

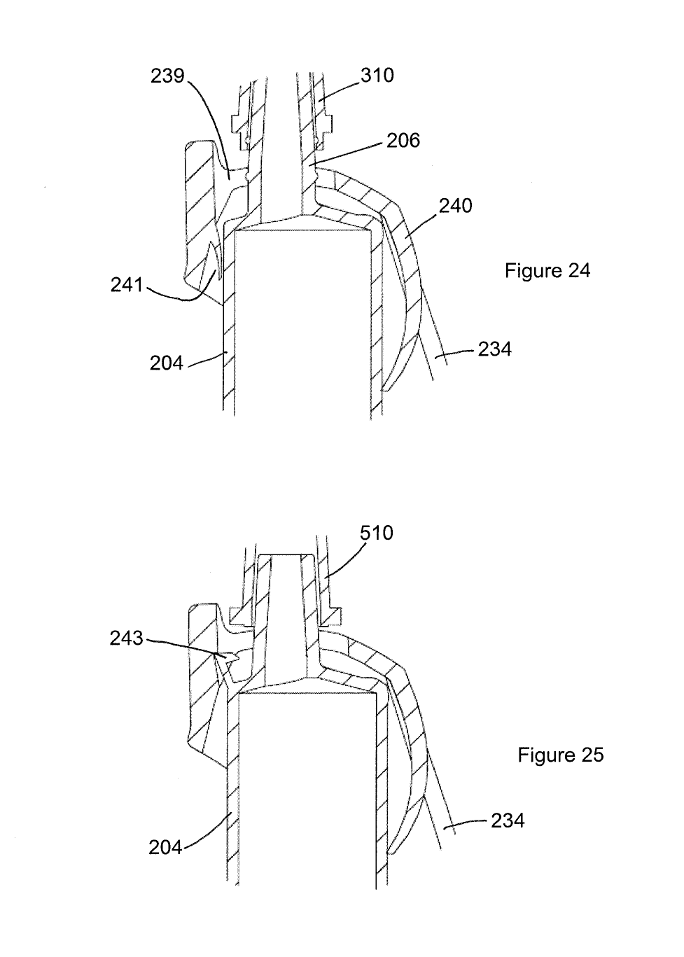

FIGS. 24 and 25 show some optional variants;

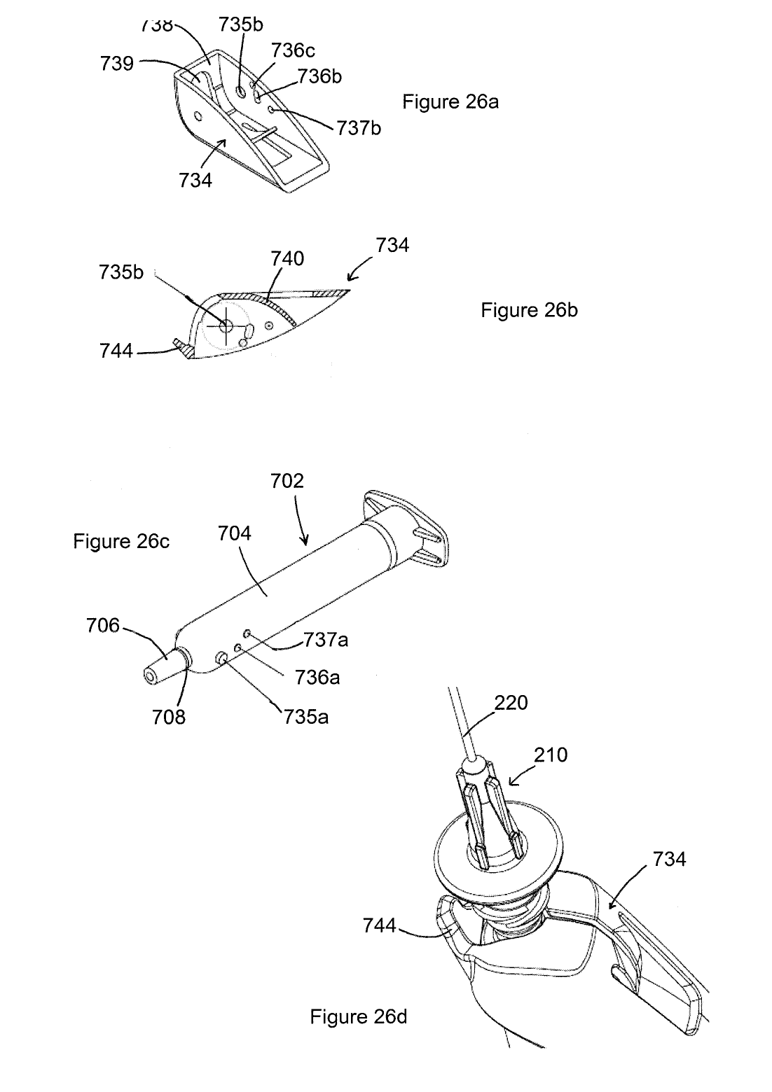

FIGS. 26a to 26d show a tenth embodiment of a disconnecting mechanism for a syringe showing connection to the fourth needle hub;

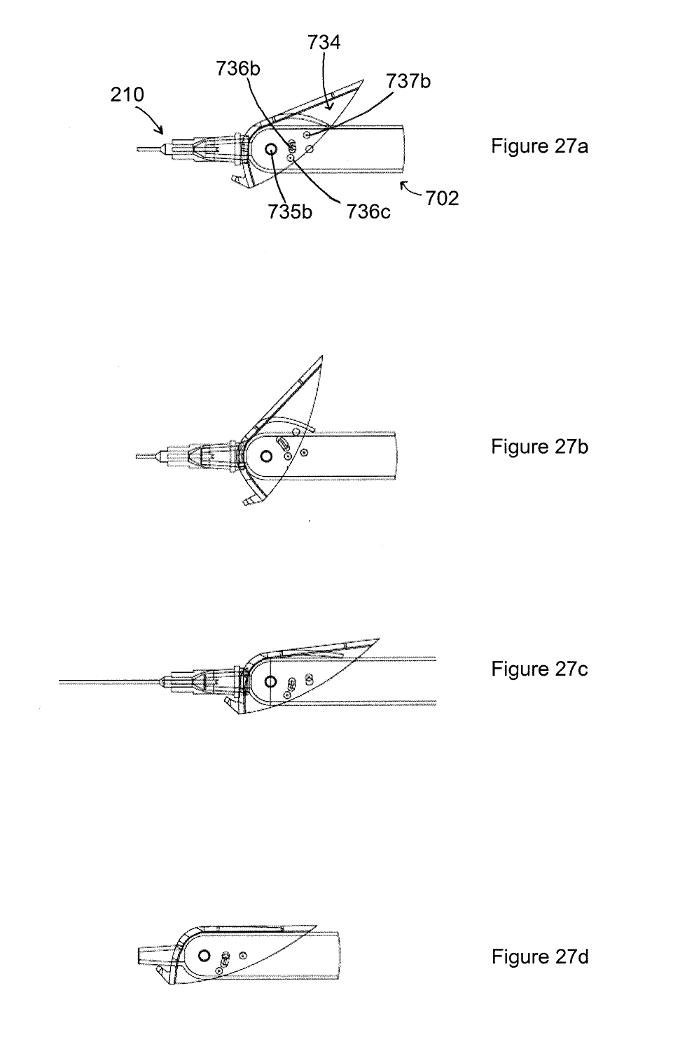

FIGS. 27a to 27d show various stages of operation of the tenth embodiment of a disconnecting mechanism;

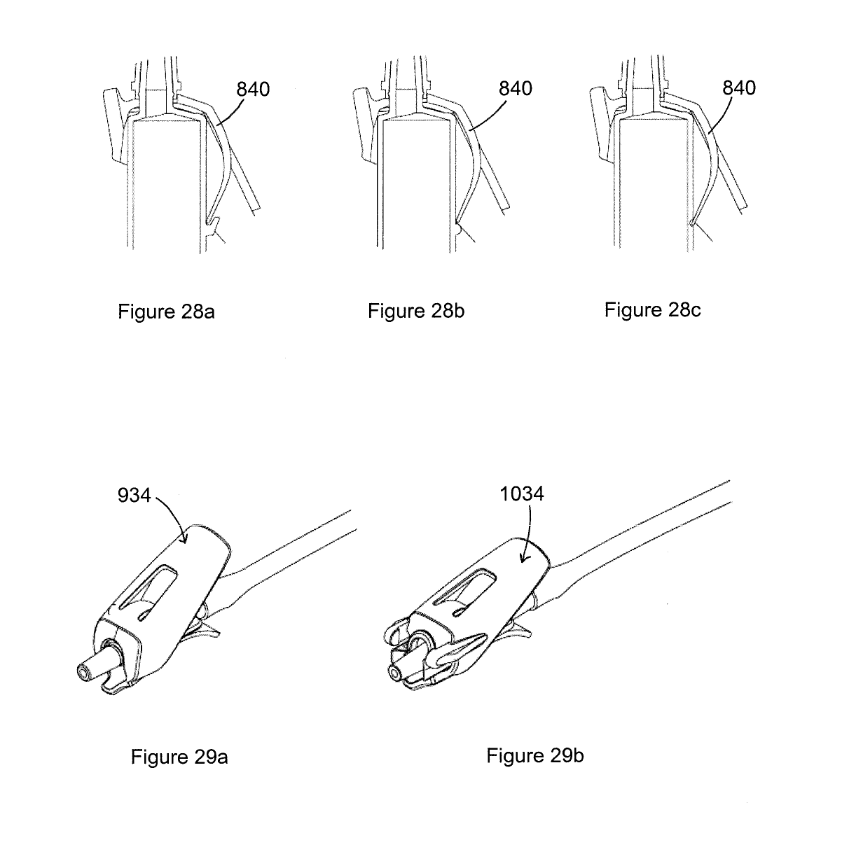

FIGS. 28a to 28c show some optional variants;

FIGS. 29a and 29b show two different disconnecting mechanisms mounted to a fluid transfer tip at the end of a fluid transfer hose;

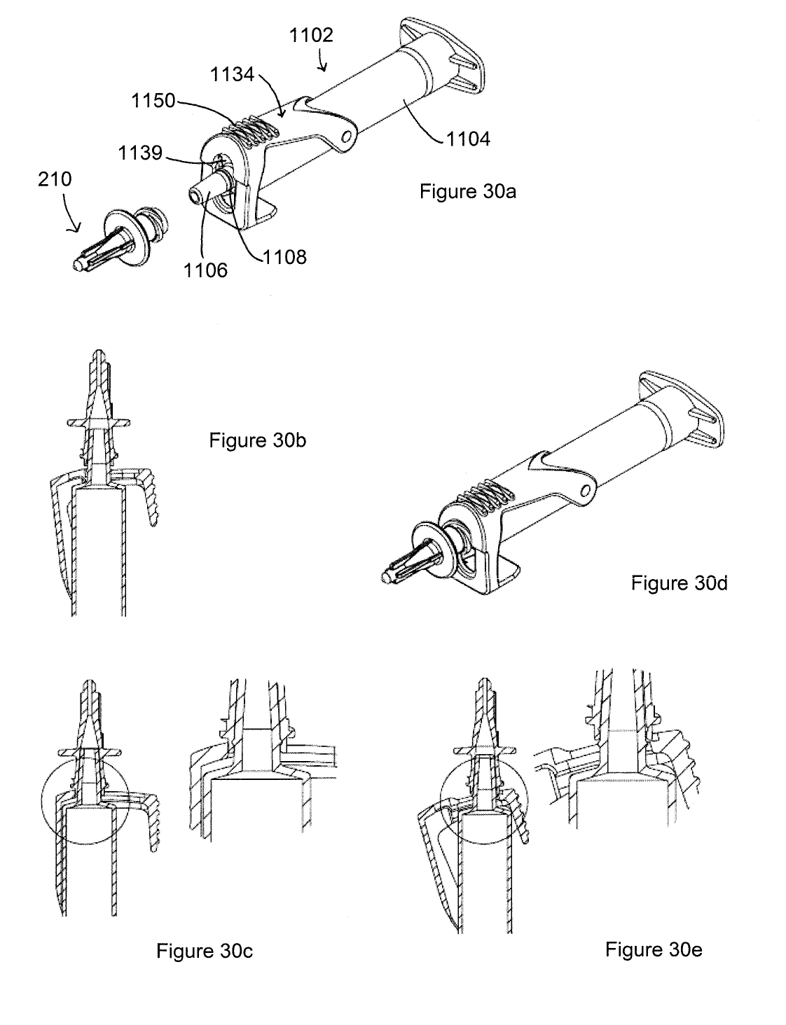

FIGS. 30a to 30e show an eleventh embodiment of a disconnecting mechanism for a syringe showing connection to the fourth needle hub;

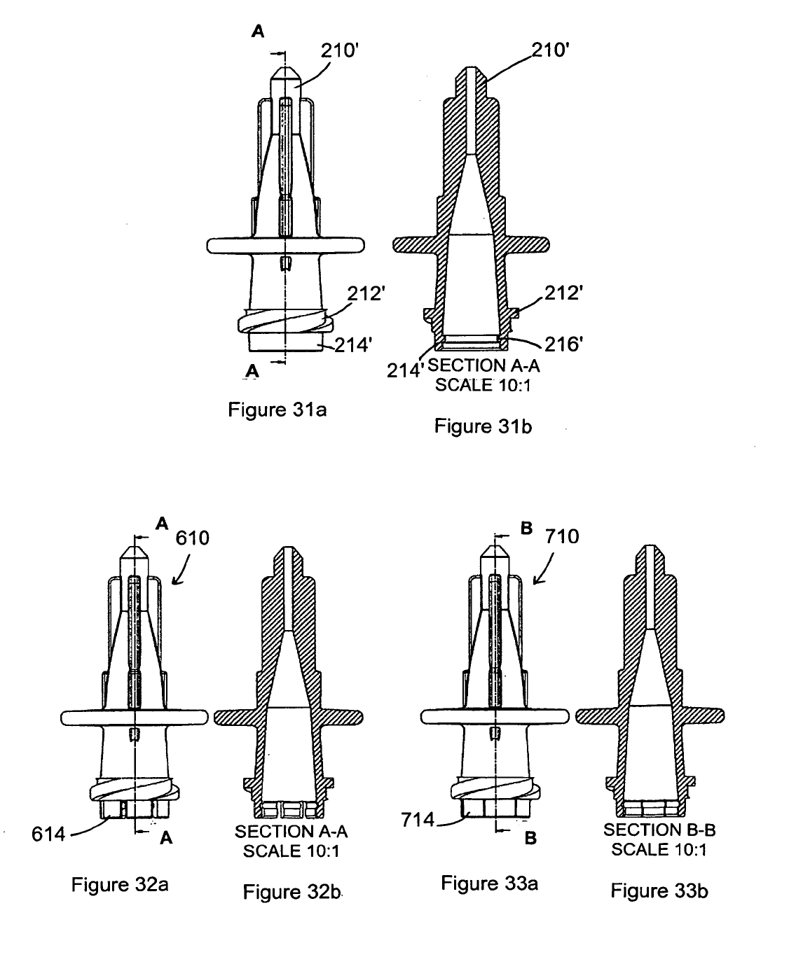

FIGS. 31 and 31b show an alternative hub;

FIGS. 32a and 32b show another alternative hub;

FIGS. 33a and 33b show another alternative hub;

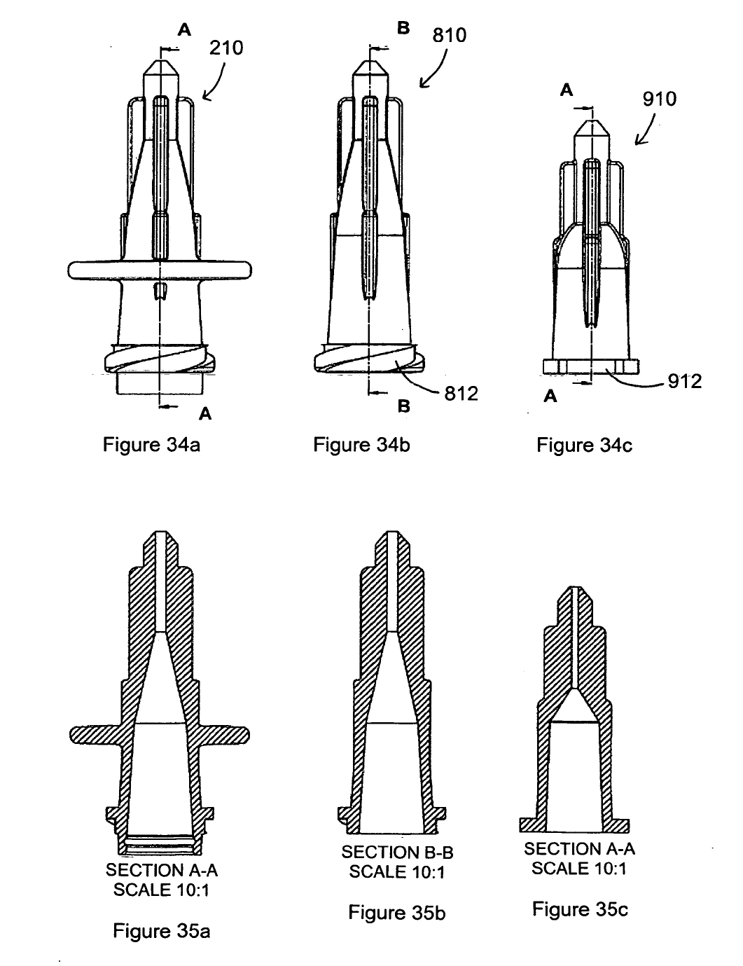

FIGS. 34a to 34c and 35a to 35c compare the fourth needle hub to conventional Luer lock and Luer slip hubs; and

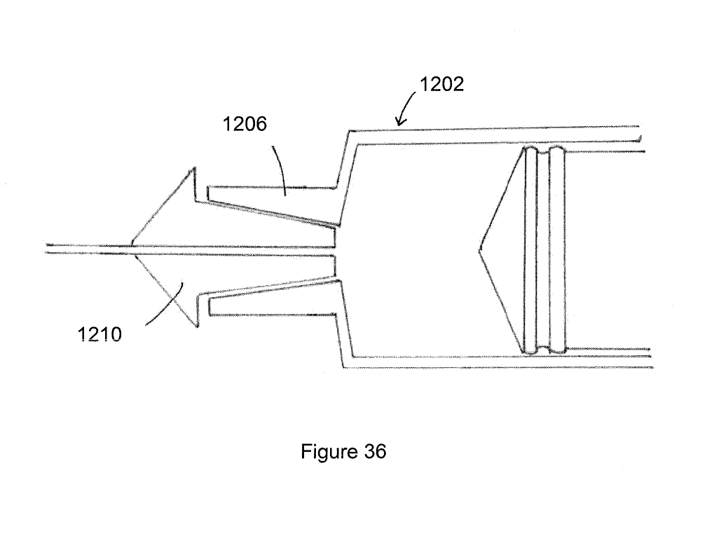

FIG. 36 shows an alternative syringe design with a female connector tip that is tapered to form a friction fit when a corresponding male hub is inserted therein.

FIGS. 1-28 and 30 illustrate various different disconnecting mechanisms for a fluid transfer device taking the form of a syringe. The syringe generally comprises a fluid barrel in communication with a male tip. The tip is tapered from its aft end, proximal to the barrel, to its forward end according to the standard Luer slip design i.e. a 6% taper (equivalent to around) 3.43°). Fluid in the barrel can be transferred through the tip by pushing or pulling a plunger inserted in the barrel. However, although a syringe is shown in each of the embodiments for simplicity, such a Luer slip tip could equally be in communication with another fluid transfer device such as a drip.

As is seen throughout the Figures, the male tip may be connected to a corresponding female hub in order to transfer fluid to a needle or other cannula. Although not shown, the needle might already be inserted into a living subject, for example for IV therapy with the hub providing an IV port for the injection and/or removal of various fluids. The tapered tip is inserted into the hub and forms a friction fit that is fluid-tight. In each of the embodiments, a disconnection mechanism is provided that can be manually operated to move relative to the male tip between a first position, proximal to the syringe barrel, and a second position spaced from the first position towards the forward end of the male tip so as to push against the hub. Operation of the mechanism therefore acts to automatically disconnect the syringe from the hub without a user needing to pull or tug the syringe to release the friction fit of the Luer slip connection. Preferred embodiments provide a lever mechanism to push the tip away from a hub.

In an embodiment seen in FIGS. 1a to 1h, a syringe 102 is provided both with a locking system 140 for a lever 34 and also a way to grip a needle hub 10 when it is connected thereto. As seen in FIGS. 1a and 1b, a pair of gripping fingers 100 is provided at a front end of the syringe barrel 104, extending forwards parallel to the tapered tip 106. The gripping fingers 100 are integrally moulded with the syringe barrel 104. The lever 34 is designed to accommodate the gripping fingers 100 with additional apertures 101 provided in its front surface. The lever 34 can be mounted on pivot axles 35 provided by the syringe barrel 104. From the cross-sectional views of FIG. 1c it can most easily be seen that the fingers 100 engage with an inside surface of the needle hub 10 when it is connected to the tip 106. The fingers 100 have outwardly facing teeth to grip inside the hub 10. The hub 10 is not a standard needle hub for a Luer slip connection but one designed for use with the gripping system.

Operation of the syringe 102 is shown in detail in FIGS. 1d-1h. In FIG. 1d it is seen that mounting the needle hub 10 by pushing it along the tip 106 towards the barrel 104 causes the fingers 100 to be pushed inwards. The hub 10 may therefore be connected smoothly without interference from the fingers 100. When the needle hub 10 is fully connected, as shown in FIG. 1e, the fingers 100 engage with the hub 10 and may be accompanied by an audible “click” or other sound. The force of connecting the hub 10 pivots the lever 34 so that it is released from the latch members 140 of the locking system, which may also be accompanied by an audible “click” or other sound. The lever 34 is now primed and may be used at any time to aid with disconnection. After the syringe 102 has been used, it can be disconnected from the needle hub 10 by pressing the lever 34 back towards the syringe barrel 104, as is seen in FIG. 1f. As the lever 34 pivots, it pushes the fingers 100 inwardly to release their grip on the hub 10. FIGS. 1g and 1h show that as the lever 34 is pressed all the way down against the syringe barrel 104, the hub 10 is pushed off the tip 106 and the lever 34 is again locked by the latch members 140. It is an optional feature for the barrel 104 to provide a locking system 140 for the lever 34.

Another embodiment of the syringe 102 is seen in FIGS. 2a-2e. The syringe 102 is adapted to additionally grip a needle hub 110 that has a standard Luer slip connection. In this embodiment a pair of gripping fingers 100′ is designed to snap-fit onto the needle hub 110 when it is connected to the syringe 102. The gripping fingers 100′ therefore have teeth arranged to grip onto the outside of the hub 110 rather than being arranged for an internal engagement e.g. seen in FIGS. 1a-1h. One or more rows of teeth may be provided. The hub 110 may be provided with a rim or other surface to facilitate engagement of the fingers 100′. As before, the lever 34 is provided with apertures 101 that accommodate the gripping fingers 100′. FIG. 2c shows the syringe 102 connected to the hub 110 with the gripping fingers 100′ engaged to hold the hub 110 in addition to the friction fit of the Luer slip connection. FIG. 2d shows how pressing the lever 34 causes the fingers 100′ to be pushed outwardly to release their grip while the hub 110 is pushed along the tip 106. The syringe 102 may also be connected to a standard needle hub 110′ that does not allow the gripping fingers 100′ to engage with its outer surface while the operation of the lever 34 remains unchanged, as is illustrated by the alternative version seen in FIG. 2e.

FIGS. 3 and 4 illustrate another embodiment in which a lever mechanism comprises two members arranged to pivot so as to provide an overall movement that is substantially aligned with the axis of the syringe tip 6. In this embodiment a sliding sleeve 94 is arranged coaxially on the syringe barrel 4 to actuate the lever mechanism. However, it will be appreciated that in this embodiment the lever mechanism could be actuated directly and the slider 94 could be omitted. As is seen from the various views provided by FIGS. 3a, 3b and 3c, the lever mechanism comprises two lever members 98 pivotally mounted at the front of the syringe 2. Advantageously, the lever members 98 can be integrally moulded in one piece with the barrel 4 of the syringe 2, with living hinges to enable pivoting of the lever members 98. The lever members 98 are recessed inside a support wall 97 that helps to ensure that they move linearly along the axis of the tip 6 when pushed by the slider 94. A cap 13 for the needle 12 is also shown in FIG. 3a. An optional feature of this embodiment is a pair of snap-fit fingers 100″, or other gripping means, that positively engage i.e. grip a needle hub 110′ when it is connected to the tip 6. The hub 110′ is provided with a ledge or other surface for the snap-fit fingers 100″ to grip. Although in this embodiment the fingers 100″ are arranged to grip onto an outer surface of the hub 110′, it will be appreciated that they could instead engage inside the hub 110′ in a similar manner to the mechanism described for FIG. 1 above.

Operation of the syringe 2 is shown in FIGS. 4a to 4d. From FIG. 4a it can be seen that mounting the needle hub 110′ by pushing it along the tip 6 of the syringe 2 causes the snap-fit fingers 100″ to be pushed apart. As is seen from FIG. 4b, when the needle hub 110′ is fully connected to the syringe 2 the fingers 100″ snap into place to hold the hub 110′ in position. An audible “click” or other noise may indicate that the hub 110′ has been pushed on far enough for it to be positively gripped. This provides an additional level of safety as it means that the needle 12 and hub 110′ can not be accidentally pulled free from the friction fit with the tip 6. FIG. 4c shows how the needle hub 110′ can be disconnected from the syringe 2 after use. As the slider 94 is pushed forwards, the lever members 98 pivot about their living hinges to push against the needle hub 110′. At the same time, as is seen from the close-up detail, pivoting of the lever members 98 causes the fingers 100″ to flex outwards so that the hub 110′ is released from their grip. FIG. 4d shows the needle hub 110′ being pushed forwards along the tip 6, with the symmetrically arranged lever members 98 acting to provide an overall force that is aligned with the axis of the tip 6 so that there no turning force is being applied. If, for example, the needle 12 is inserted into a patient then it can be disconnected from the syringe 2 smoothly without any twisting that could cause damage.

A further feature of this, and other embodiments, is that the sleeve 94 (or other part of a disconnecting mechanism) can be arranged to render the device unusable after the hub 110′ has been disconnected. This may be achieved, for example, by forward movement of the sleeve 94 crushing or severing the lever members 98 so that they are no longer operative. This ensures that the device is single use only.

There is seen in FIGS. 5a and 5b a further embodiment of a syringe 202 comprising a barrel 204 connected to a tapered fluid transfer tip 206. A lever member 234 is pivotally mounted to an axle 235 provided at a front end of the barrel 204. The lever member 234 is resiliently biased by a leaf spring 240, which may be a separate spring but in this embodiment is integrally moulded with the lever member 234. The lever member 234 is shaped to generally match the cylindrical surface of the syringe barrel 204. The lever member 234 has a front surface 238 that is substantially transverse to the axis of the tapered tip 206 and cylindrical side surfaces 236 that extend in a direction generally parallel to the axis of the tip 206. The cylindrical shape of the lever member 234 imparts stiffness so that it is able to transmit forces without flexing. The lever 234 may be moulded from a plastics material. The forward surface 238 of the lever member 234 is provided with a slot 239 to accommodate the tip 206 of the syringe 202. A catch member 244 is provided next to the slot 239. As is seen from FIG. 5b, when the lever member 234 is assembled onto the barrel 204, the tapered tip 206 protrudes through the slot 239 in the front surface 238 of the lever member 234 so as to receive a corresponding hub 210 that may carry a needle (not shown) or other cannula. From FIG. 5a it can be seen that the syringe 202 differs from a standard syringe in that it has an axle 235 moulded onto the barrel 204, and in addition the tapered fluid transfer tip 206 is provided with an annular gripping flange 208 surrounding the tip 206 close to the barrel 204.

The design of the hub 210 is seen in more detail in FIGS. 5c and 5d. From FIG. 5c it may be seen that the hub 210 carries an external thread 212, which means that the hub 210 may be used with a standard Luer lock connection if desired. The thread 212 may be omitted or replaced by a plain rim, in other variants. However, as compared to a conventional Luer lock hub (seen in FIGS. 33b and 34b), the hub 210 comprises a skirt 214 below the thread 212. The skirt 214 extends downwardly so as to pass through the slot 239 in the front surface 238 of the lever member 234. The skirt 214 therefore provides a surface that helps the lever member 234 to engage the hub 210, as will be described in more detail below. It can further be seen from the cross-section shown in FIG. 5d that the skirt 214 has an annular groove 216 formed on its inner surface in addition to being tapered. The groove 216 provides an additional means for the hub 210 to be gripped in a locked position when connected to the fluid transfer tip 206 by a friction fitting. Finally, it can be seen from FIGS. 5c and 5d that the hub 210 may optionally include an outer ring 218 which is an ergonomic feature making it easier for a user to push the hub 210 onto a tip 206. The syringe 202 is also compatible with existing hub designs, for example a standard Luer slip or Luer lock hub without the internal groove to provide for additional gripping onto the tip 206.

Operation of the disconnecting mechanism for the syringe 202 will now be described with reference to FIGS. 6 to 12. Firstly, FIGS. 6a to 6c show the hub 210 being initially connected onto the tapered fluid transfer tip 206 of the syringe 202. Due to the resilient bias of the leaf spring 240, the lever member 234 automatically adopts a position in which the slot 239 does not provide enough space around the tip 206 for the hub 210. In this embodiment the lever member 234 comprises a nose 242 that extends forward parallel to the fluid transfer tip 206. The nose 242 has an angled forward surface arranged to be contacted by the outer thread 212 of a hub as it is initially pushed along the tapered transfer tip 206. As the hub 210 starts to connect to the tip 206, it pushes along the angled forward surface of the nose 242, acting to temporarily pivot the lever member 234 against the resilient bias of the leaf spring 240 and therefore allow the hub 210 to slide down the tapered tip 206, as is seen in FIGS. 6b and 6c. The nose 242 therefore automatically operates the lever member 234. In other versions of the syringe 202, the nose 242 may be omitted and the lever member 234 manually operated to open up the slot 239 for connection.

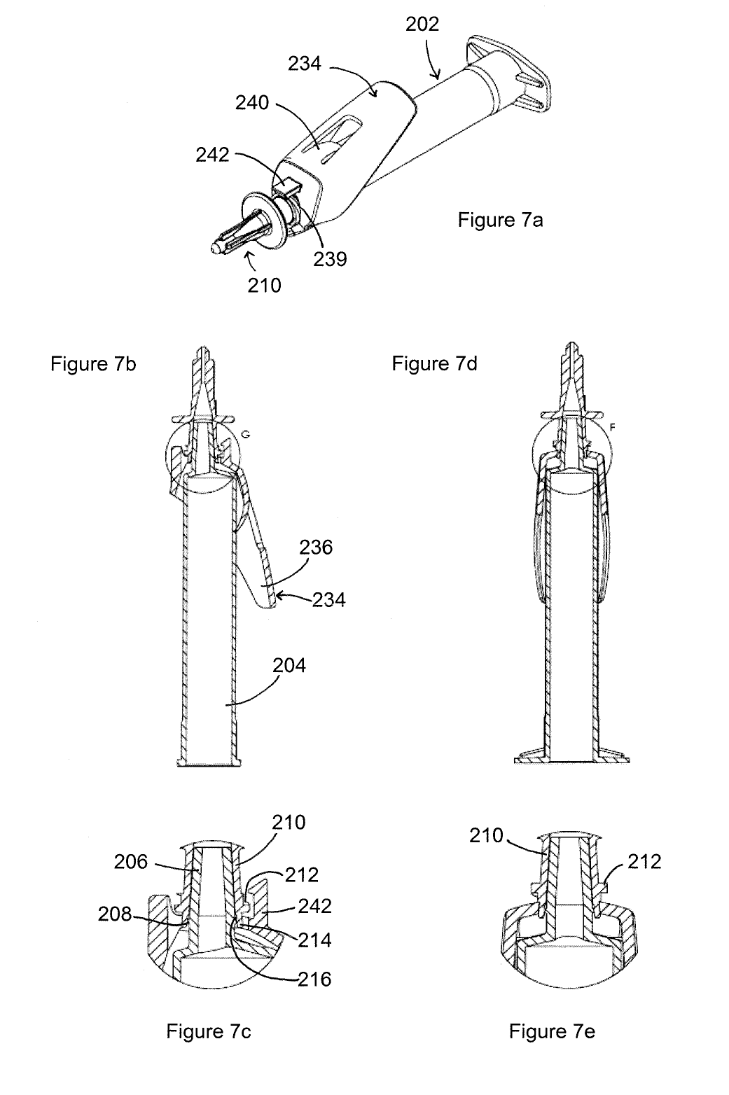

FIGS. 7a to 7e show how the hub 210 becomes additionally gripped in a locked position as it is fully connected to the fluid transfer tip 206 with the normal friction fitting. The groove 216 on the inside of the hub 210 engages over the corresponding annular flange 208 provided towards the bottom of the tip 206. The interlocking of the flange 208 with the groove 216 may provide an audible “click” when the hub 210 is fully connected to the syringe 202.

From FIG. 7b, and the close-up of FIG. 7c, it can be seen that as the hub 210 slides further down the tip 206, the outer thread 212 moves past the angled forward surface of the nose 242 and therefore releases the lever member 234 so that its side surface 236 can pivot away from the syringe barrel 204 under the resilient bias of the leaf spring 240. As is seen from FIG. 7d and the close-up of FIG. 7e, the movement of the lever member 234 means that as the hub 210 engages its groove 216 over the flange 208, the side surfaces of the slot 239 move into contact with the skirt 214 so as to press the groove 216 against the flange 208 and thereby assist in holding the hub 210 in the locked position. A “double locking” effect is assured by the resilient bias acting on the lever member 234.

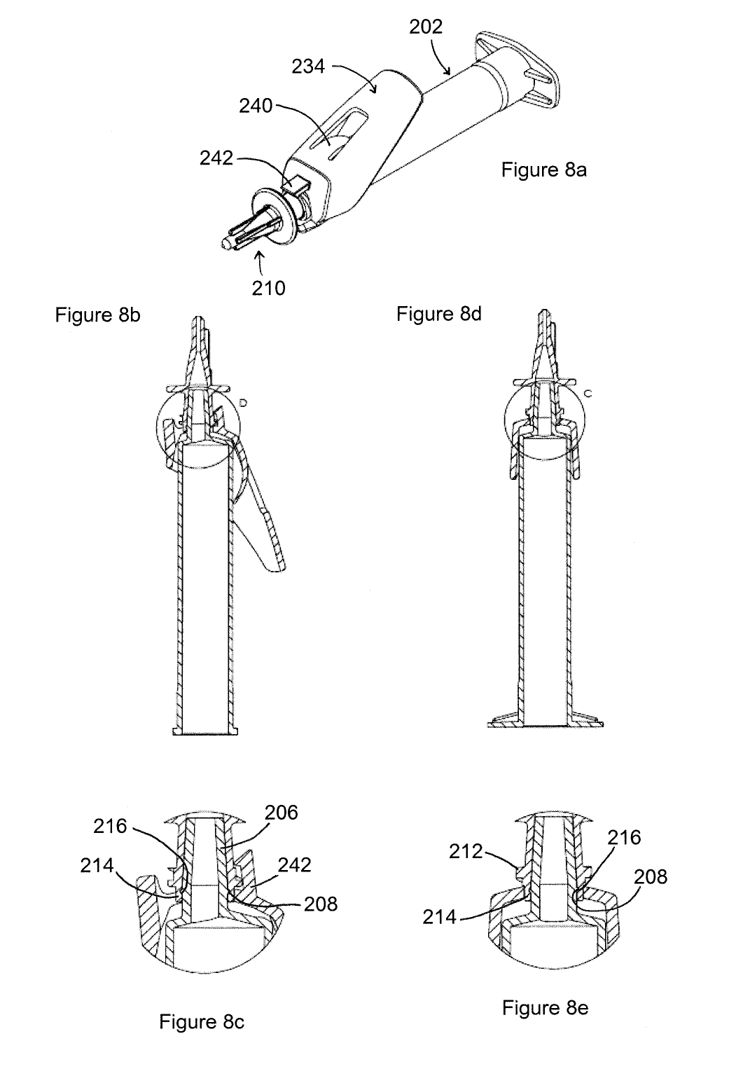

The final locked position of the hub 210 on the fluid transfer tip 206 is shown in FIGS. 8a to 8e. From the cross-sections of FIGS. 8b and 8d, and in particular the close-ups seen in FIGS. 8c and 8e, it is seen that the groove 216 on the inside of the hub 210 engages over the flange 208 so that the hub 210 is gripped onto the tip 206 in addition to the normal friction fitting between the tapered surfaces. Furthermore, it can be seen that the lever member 234 has pivoted back under the resilient bias of the leaf spring 240 so as to apply physical pressure around the skirt 214 of the hub 210 and thereby assist in holding the hub 210 in the locked position. With the hub 210 firmly attached to the syringe 202 in the locked position, there is no risk of the hub 210 accidentally being pulled free. The syringe 202 may be used to provide a fluid transfer procedure for as long as necessary.

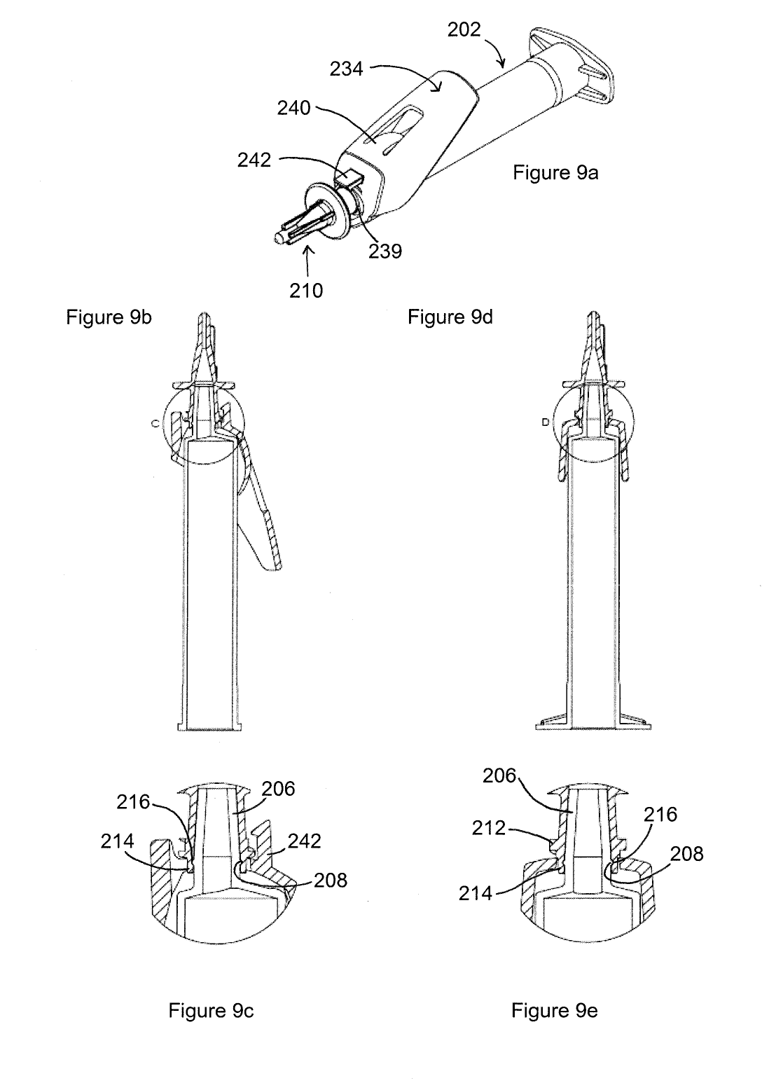

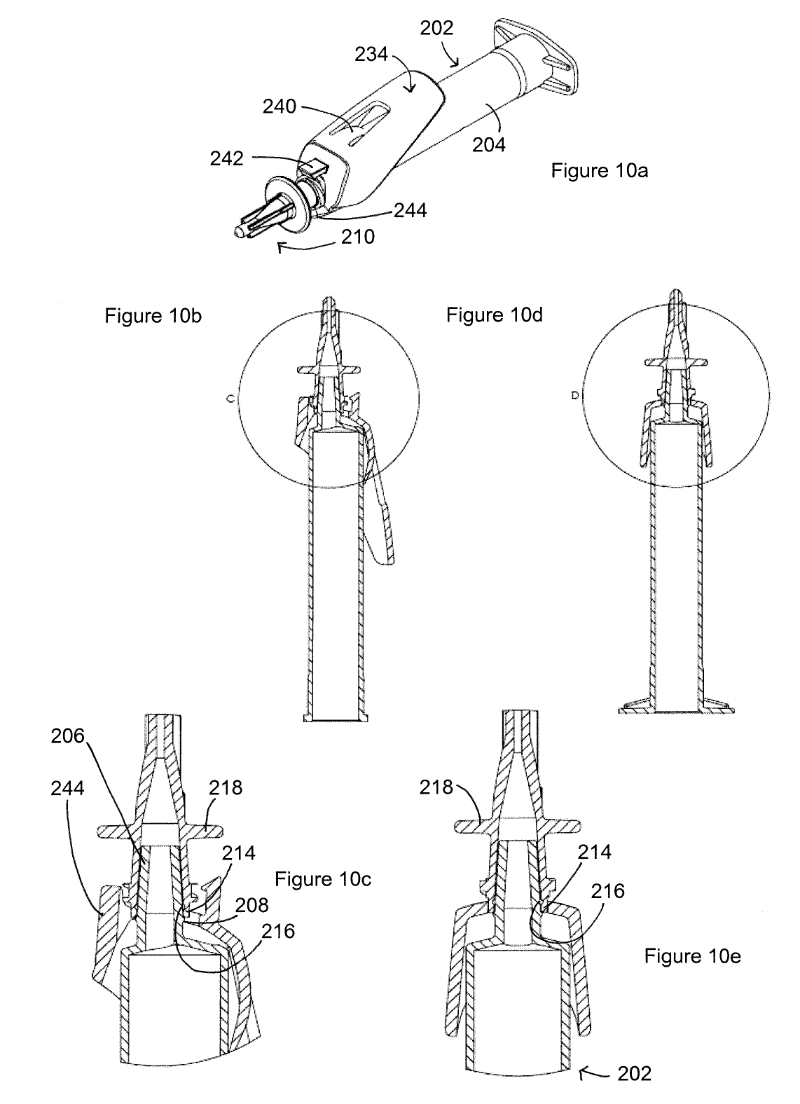

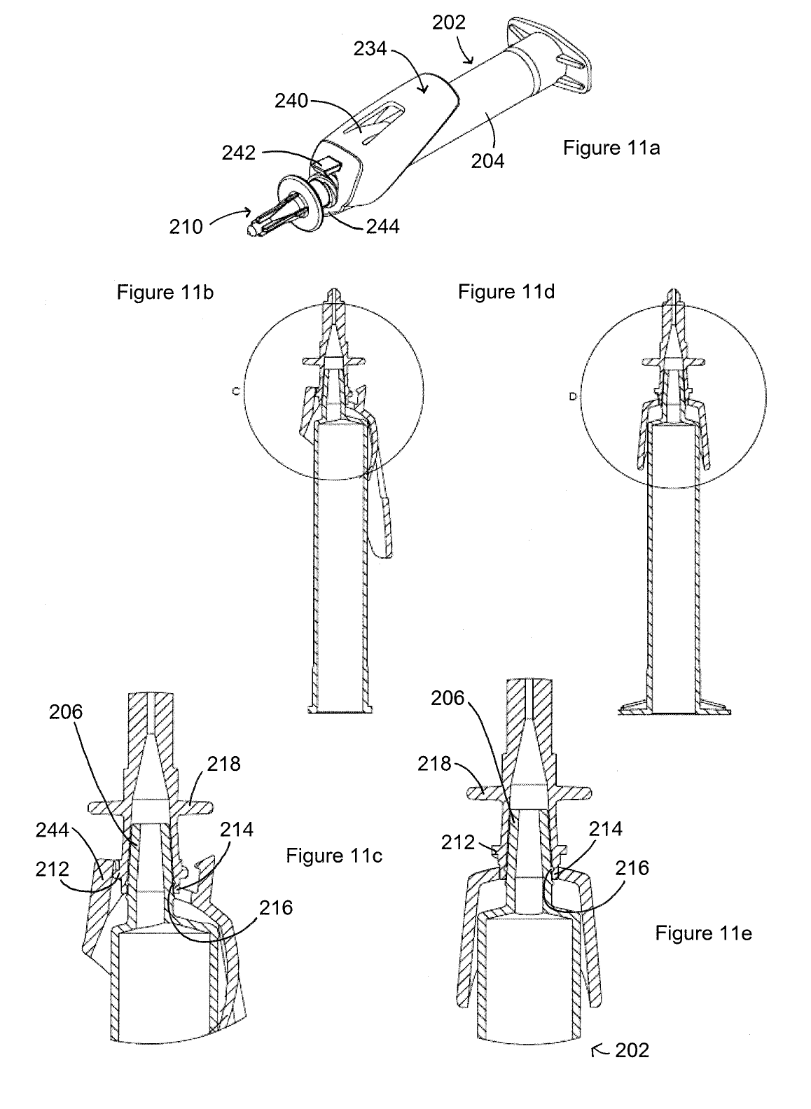

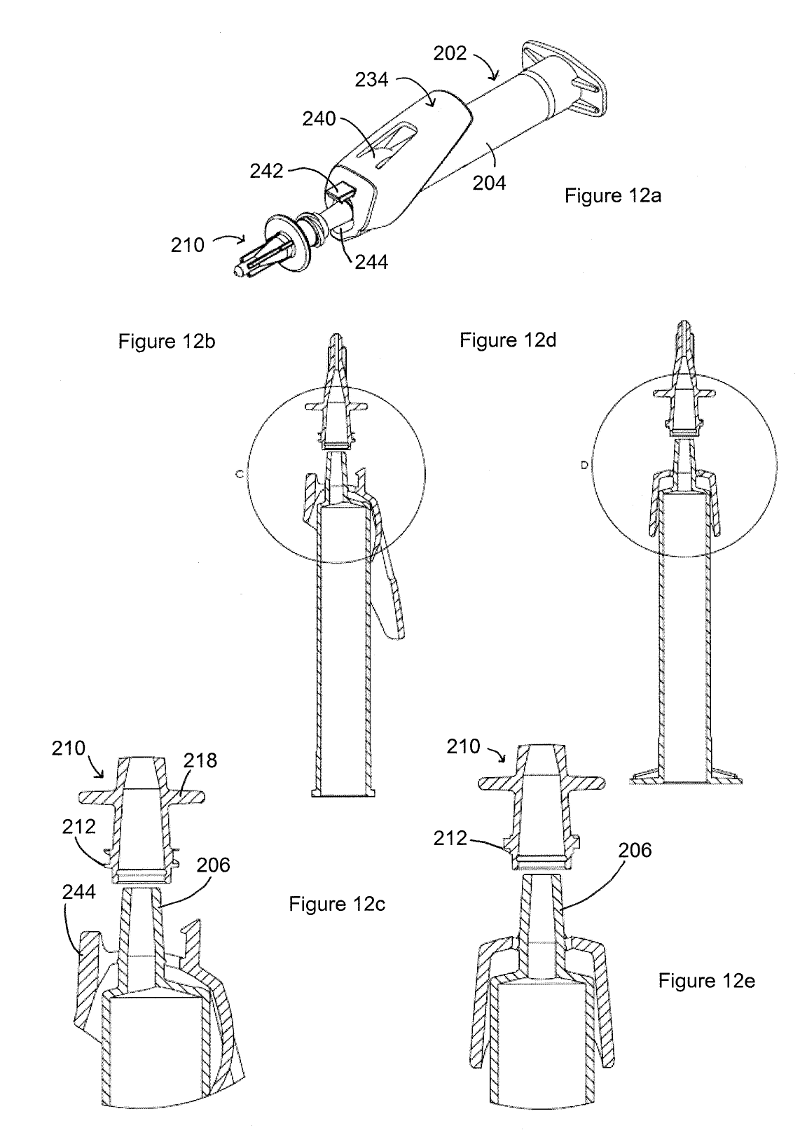

Once a procedure has been completed and it is desired to disconnect the hub 210 from the tip 206 of the syringe 202, there are three stages to the disconnection process as shown in FIGS. 9, 10 and 11. FIGS. 9a to 9e show a first stage in which the lever member 234 starts to be pivoted down against the resilient bias of the leaf spring 240. This opens up a space in the slot 239 around the hub 210 so that the skirt 214 is no longer pressed in to hold the hub 210 in its locked position. FIGS. 10a to 10e show a second stage in which the lever member 234 is pivoted further towards the barrel 204. The front surface 238 of the lever member 234 pushes forwards against the underside of the thread 214 so as to move the hub 210 out of the locked position by pushing the groove 216 out of engagement over the flange 208. At the same time, it can be seen that the catch member 244 provided on the front surface 238 of the lever member 234 starts to pivot towards the tip 206. In a final stage of disconnection seen in FIGS. 11a to 11e, the hub 210 is pushed further forwards along the tip 206 so as to be released from the friction fitting of the tapered surfaces. In order to prevent the hub 210 from flying away from the syringe 202, the catch member 244 pivots back far enough to catch onto the thread 212, or other surface, of the hub 210. As long as a user is holding the lever member 234 against the barrel 204 in this position, the catch member 244 prevents the hub 210 from being fully pushed away from the tip 206 by virtue of its own frictional engagement. Finally, in order to release the hub 210 in a controlled manner, it can be seen from FIGS. 12a to 12e that a user releases the lever member 234 so that it pivots away from the barrel 204 under the resilient bias of the leaf spring 240. As the lever member 234 pivots back, the catch member 244 swings away from the hub 210 so that the hub 210 is free to fall away. The syringe 202 has now returned to its starting configuration and may be re-used, if hygiene standards allow, to re-connect the same hub or connect another hub.

There is seen in FIGS. 13a to 13e an alternative embodiment of the syringe 202 using a different lever member 234′. As before, the lever member 234′ is pivotally attached to the syringe barrel 204. It can be seen that the lever member 234′ still has a generally cylindrical shape so as to fit over the barrel 204, but with straight sides 236 that give it a rectangular cross-section. It can also be seen that the front surface 238 of the lever member 234′ is continuously curved so as to extend back into the main sidewall 236. This lever member 234′ does not have a nose extending forwards to be actuated by the hub 210 when it is first pushed onto the tip 206. Instead, the front surface 238 is provided with a shaped slot 239 that provides space around the tip 206 depending on the position of the lever member 234′. The slot 239 has side surfaces that are angled so that the skirt 214 of the hub 210 can be pushed down into the slot 239. Furthermore, it can be seen from the perspective view of FIG. 13a, and the front end view of FIG. 13f, that the slot 239 varies in diameter, with a narrower space provided around the tip 206 when the lever member 234′ is pivoted fully under the resilient bias of the leaf spring 240.

FIGS. 13b and 13c show the hub 210 being pushed down the tip 206. A wedging action between the skirt 214 and the side surfaces of the slot 239 forces the lever member 234′ to pivot towards the barrel 204 and make space for the hub 210 to be pushed onto the tip 206. This may be assisted by a user manually pressing down the lever member 234′. The skirt 214 is elastic enough to expand as it is pushed over the flange 208 towards the bottom of the tip 206. As is shown in FIGS. 13d and 13e, once the groove 216 on the inside of the hub 210 engages over the flange 208, the skirt 214 is able to move back in towards the tip 206 and therefore pressure is released from the side surfaces of the slot 239. The lever member 234′ therefore pivots back under the resilient bias of the leaf spring 240 to a locking position seen from the front view of FIG. 13f. In this position, the narrower diameter of the slot 239 surrounds the hub 210 and therefore acts to compress the skirt 214 and provide a tight fit surrounding the gripping arrangement of the groove 216 over the flange 208.

The hub 210 may be disconnected from the tip 206 by operating the lever member 234′ in the same way as is described above with respect to FIGS. 9 to 12. However, a different feature of the front surface 238 is that it has a varying curvature. When the lever member 234′ is first pivoted against the resilient bias of the lea spring 240, the curvature of the front surface 238 is such that it moves substantially transverse to the axis of the tapered tip 206. This allows the locking position to be released from the tight grip of the slot 239 without any force being applied to push away the hub 210. As the lever member 234′ is pivoted further, the curvature of the front surface 238 is such that it starts to move forwards along the tip 206 so as to push the hub 210 out of the locked position and to release it from the friction fitting. The curvature of the front surface 238 therefore provides for two separate stages of movement without interference between them. As the hub 210 is released, the catch member 244 may operate as previously described.

FIGS. 14 and 15 show some alternative variants for the lever member 234′ of FIG. 13. In FIGS. 14a and 14b, it may be seen that the slot 239′ is provided with a keying ridge 250 on at least one side surface, and the hub 210′ provides a corresponding groove on the outer surface of the skirt 214′. As can be seen from FIG. 14b, when the lever member 234′ is pivoted back under its resilient bias, the keying ridge 250 intersects with the groove on the skirt 214′ of the hub 210′ so as to provide additional locking between the hub 210′ and the lever member 234′ in this position. In a further alternative variant seen in FIGS. 15a and 15b, the slot 239″ in the front surface 238 of the lever member 234′ has an angled or wedged side surface 250′ that slides against the outer surface of the skirt 214 to frictionally lock the hub onto the tip whenever the lever member 234′ is pivoted fully under its resilient bias. The side surface features 250′ may be designed to interact with the skirt 214 and/or thread 212 on the outside of the hub 210.

There is seen in FIGS. 16 and 17 another embodiment of a syringe 302 that uses a different disconnecting mechanism. As shown in FIGS. 16a and 16b, in this embodiment a lever member 334 is again pivotally mounted to the barrel 304 of the syringe 302 by an axle 335. The lever member 334 is resiliently biased by a leaf spring 340 that engages against the side of the barrel 304. The syringe 302 has a fluid transfer tip 306 that is tapered so as to form a Luer slip connection i.e. friction fitting with a corresponding hub 310. The hub 310 is similar to a standard Luer slip hub having an internal taper and an outer rim 312, except that this hub 310 is additionally provided with a skirt 314 extending below the rim 312. FIGS. 16c and 16d show a side view and cross-sectional view, respectively, of a standard Luer slip hub for comparison.

The syringe 302 includes a pair of gripping fingers 320 provided at a front end of the barrel 304, and extending forwards parallel to the tapered tip 306. The gripping fingers 320 are integrally moulded with the barrel 304. The gripping fingers 320 can be flexed outwardly from the tip 306 if a force is applied to an actuator e.g. knob 321 on their inwardly facing surface. The front surface 338 of the lever member 334 includes both a central slot 339 for the tip 306 and a pair of additional apertures either side of the slot 339 that accommodate the gripping fingers 320 passing therethrough, as is seen in FIG. 16b. The slot 339 is surrounded on its top side by an actuating lip 323 that will press down on the actuators 321 if the front surface 338 is pivoted forwards. The gripping fingers 320 can engage over the rim 312 of the hub 310 so as to hold the hub 310 in a locked position when it is also connected to the fluid transfer tip 306 by a friction fitting. The lever member 334 is provided with a pair of forwardly extending support fingers 322 that can act to engage the gripping fingers 320 and thereby assist in holding the hub 310 in the locked position.

Operation of the disconnection mechanism for the syringe 302 will now be described with reference to FIGS. 17a to 17i. FIGS. 17a to 17c show a first stage of connection where the lever member 334 is operated at least partway against the resilient bias of the leaf spring 340 so that the support fingers 322 are not engaging the gripping fingers 320. As the hub 310 is initially pushed down onto the tapered tip 306, the rim 312 on the outside of the hub 310 slides down the angled front surface of the gripping fingers 320. In this position the slot 339 provides plenty of space around the tip 306 to accommodate the skirt 314 of the hub 310. FIGS. 17d to 17f show the locked position where the hub 310 is fully connected with the fluid transfer tip 306. This can be seen from the cross-section of FIG. 17e, and in particular the close-up view of FIG. 17f. Moving into this position allows the support fingers 322 to slide against the outer surface of the gripping fingers 320 as the lever member 334 naturally pivots under the resilient bias of the leaf spring 340. In this position, the support fingers 322 press against the gripping fingers 320 to push them inwardly so as to assist in holding the hub 310 in the locked position. With the lever member 334 biased in this position, the hub 310 can not be pulled loose from the gripping fingers 320.

The stages of disconnection are illustrated with reference to FIGS. 17g to 17k. FIG. 17g shows the lever member 334 being pressed down against the barrel 304 of the syringe 302, against the resilient bias of the leaf spring 340. As the lever member 334 pivots, the support fingers 332 are moved away from their engagement with the gripping fingers 320. This may allow the gripping fingers 320 to flex outwardly of their own accord, but the gripping fingers are actively released by the actuating lip 323 pressing down on the actuators 321 to push the gripping fingers 320 open (see cross-section of FIG. 17h). Once the gripping fingers 320 are no longer engaged over the rim 312 of the hub 310, the hub 310 is released from its locked position. As the lever member 334 moves against the resilient bias of the leaf spring 340, its front surface 338 moves forwards along the tip 306 to push the hub 310 away from the barrel 304. As is seen from the cross-section of FIG. 17i, the hub 310 may be prevented from being pushed away entirely by the catch member 344 which frictionally engages the hub 310 when the lever member 334 is squeezed down against the barrel 304. FIGS. 17j and 17k show a final stage of disconnection where the lever member 334 is released so as to pivot automatically under the resilient bias of the leaf spring 340, thereby moving away the catch member 344 so that the hub 310 is free to fall away from the syringe 302.

FIGS. 18a to 18c show some variants of a syringe 302′ using gripping fingers to additionally hold a hub in a locked position when it is connected to the fluid transfer tip by a friction fitting. In the variant of FIGS. 18a and 18b, the hub 210 is one that has an outer thread 212 rather than a rim, for example as already described above in relation to FIGS. 5c and 5d. Although the hub 210 is shown as having a skirt 214, this may be omitted and in fact the syringe 302′ is compatible with a standard Luer lock hub as well (see FIGS. 34b and 35b). So as to accommodate the outer thread 212 of the hub 210, the gripping fingers 320′ have a thread formed on their inner surface. Two-handed manipulation may be required to turn the hub 210 as it is pushed onto the tip 306, but to disconnect the hub 210 there is only required single-handed operation of the lever member 334. FIG. 18c shows an alternative version of the syringe 302′ in which the gripping fingers 320′ have a stepped inner surface rather than a threaded one.

In the embodiments of FIGS. 5 to 15, it is the side surfaces of the slot in the front surface of the lever member that press against or intersect with the skirt at the bottom of the hub so as to assist in holding the hub on the tip in a locked position provided by the engagement of the groove and flange. An alternative solution, seen in FIGS. 19a to 19c, is for the lever member 434 to include a latch member 446 that can engage with a keeper on the outer surface of the hub 310. Where the hub 310 includes an outer rim 312 rather than a thread, the latch member 446 can be arranged to hook over the rim 312 when the hub 310 is connected to the tip 406 by the friction fitting and engagement of the groove 316 over the flange 408 on the tip 406. In FIG. 20 there is seen an alternative embodiment where the lever member 434′ is provided with a pair of forwardly extending latch members 456 that engage over the outer rim 312 of the hub 310. In another variant seen in FIG. 21, the lever member 434″ is provided with an outwardly facing latch member 466 that can lock inside a different design of hub 410.

FIGS. 22a to 22d illustrate a further embodiment of a disconnecting mechanism for the hub 310. In this embodiment the syringe 502 has a pivotally mounted lever member 534 which carries a forwardly extending latch 546 in the form of a partial hemispherical collar carrying an internal thread. As is seen from FIG. 22b, the hub 310 may be connected to the tip 506 by pushing it onto the friction fitting at the same time as twisting the hub 310 to connect with the threads of the latch 546. In its resiliently biased position, seen in FIG. 22c, the lever member 534 is pivoted to keep the threads positively engaged. Although a two-handed operation may be required to connect the hub 310 to the syringe 502, the lever member 534 can be used to disconnect the hub 310 in a continuous, single-handed movement. FIG. 22d shows the lever member 534 being pivoted down against the resilient bias of its leaf spring 540 so that the threaded latch 546 is moved away from the hub 310 so that it no longer assists in holding the hub in a locked position. Pivotal movement of the lever member 534 also pushes the hub 310 out of engagement with the rim on the tip and releases the friction fitting. The optional catch member 544 is provided to prevent the hub 310 from flying away from the syringe 502.

In a further unillustrated embodiment, it is envisaged that a lever member 534 carrying such a the threaded latch 546 could be combined with a syringe 302′ as seen in FIGS. 18a and 18b having internally threaded fingers 320′. When the lever member 534 is acted on by its resilient bias to lock the gripping fingers 320′, the two sets of threads could align to surround the hub in threaded engagement. The threaded latch 546 might also be designed to act as a support for the fingers 320′.

FIGS. 23a to 23d illustrate another alternative embodiment of a syringe 602 comprising a pivoting lever member 634 and a gripping means in the form of multiple latch members or snap-fit members 646. This embodiment is shown without the benefit of a “double locking” mechanism. The hub 510 is similar to a standard Luer slip hub with a tapered inner surface and no inner groove to accommodate a corresponding flange on the tip 606 of the syringe 602. However, the hub 510 is not exactly the same as a standard Luer slip hub as it includes a skirt 514 below the rim 512. As is seen most clearly from FIG. 23b, the skirt 514 is provided with an additional rim 515 at its lower end. FIGS. 23b to 23d show the stages of connection of the hub 510 to the tapered tip 606. As the friction fitting is formed, the skirt 514 is pushed underneath the snap-fit members 646 and then securely gripped onto the tip 606. Connection may be assisted by a user manually squeezing the lever member 634 towards the barrel 604 against the resilient bias of the leaf spring 640. The snap-fit members 646 may provide an audible “click” when the hub 510 is fully connected to the syringe 602. Once the hub 510 is gripped in a locked position, the resilient bias of the leaf spring 640 causes the lever member 634 to pivot away from the barrel 604 so that an inner surface of the slot 639 in the front surface 638 of the lever member 634 engages against the snap-fit members 646 to assist in holding the hub in the locked position. The hub may be disconnected by squeezing the lever member 634 against its resilient bias in the same manner as has been described above. The snap-fit gripping members 646 therefore provide an alternative gripping mechanism to a groove and flange connection previously described.

There is seen in FIG. 24 an optional feature of any of the lever members described above. In addition to the main leaf spring 240, the lever member 234 is resiliently biased in the opposite direction by a smaller leaf spring 241. The smaller leaf spring 241 is arranged to provide a lower spring force than the main leaf spring 240, but provides sufficient bias to help open the slot 239 when a hub 310 is being connected onto the tip 206. FIG. 25 shows a further variant designed for a hub 510 where the syringe barrel 204 includes a latch member 243 that interacts with the lever member 234. The latch member 243 is actuated when the lever member 234 is squeezed towards the barrel of the syringe, so that the lever member 234 is held in a position to accommodate a hub 510. When the hub 510 is pushed down, its outer rim or thread 512 acts to open the latch member 243 so that it releases its grip on the lever member 234, allowing the lever member 234 to pivot under the resilient bias of its leaf spring.

In embodiments where a pivotally mounted lever member is resiliently biased away from the syringe barrel, it may be desirable to have some control over the position of the lever member, for example so that the lever member can more easily be put in a neutral position where the slot in its front surface provides space around the fluid transfer tip to accommodate a corresponding hub. FIGS. 26a to 26d show an embodiment of a lever member 734 which does not pivot entirely freely relative to a syringe 702. As is seen from FIG. 26a, the side surfaces of the lever member 734 have recesses formed on their inner surface. A first recess 735b is designed to engage over axle hubs 735a on the side of the syringe barrel 704. In addition, a groove 736b is arranged to run along a trajectory defined by a positioning bump 736a formed on the syringe barrel 704. Finally, a locking recess 737b can engage over a locking bump 737a on the side of the syringe barrel 704 when the lever member 734 is pressed down fully against the syringe barrel 704, e.g. for storage and/or transport. FIG. 26d shows the lever member 734 assembled on a syringe connected to a hub 210 that carries a needle 220.

FIGS. 27a to 27d show the position of the lever member 734 during various stages of connection and disconnection of the hub 210. In FIG. 27a the lever member 734 is held in an intermediate position through a combination of the leaf spring 740 and the positioning bump 736a interacting with the groove 736b. In this position the hub 210 may be pushed along the tapered tip 706 and accommodated by the slot 739 in the front surface 738 of the lever member 734. As is shown in FIG. 27b, when the hub 210 is fully connected to the tapered tip, the positioning bump 736a jumps out of the groove 736b and into a recess 736c as the lever member is pivoted away from the syringe barrel 704 by the resilient bias of the spring 740. FIG. 27c shows the lever member 734 being pressed against the syringe barrel 704 against its resilient bias. Once the hub has been fully disconnected, as is seen in FIG. 27d, the lever member 734 can be pushed down all the way to the syringe barrel 704 and the locking bump 737a therefore clicks into its corresponding recess 737b, so that the lever member 734 is locked down. When a hub 210 is again connected onto the tip 706, enough force may be applied to disengage the locking bump 737a so that the lever member 734 is released.

FIGS. 28a to 28c show three different ways of locking a leaf spring 840. The effect of locking the leaf spring 840 is to build up the spring force before he lever member operates. The lock may also be destroyed by use so that the device can not be used again.

Of course various embodiments of the present invention, such as those described above, are not limited to a fluid transfer device in the form of a syringe. It will be appreciated that the disconnecting mechanisms described herein are not limited to use with a syringe comprising a barrel as a fluid chamber, but may instead be mounted to a fluid transfer tip at the end of a hose, pipe, cannula, etc. FIGS. 29a and 29b show two different lever-actuated disconnection mechanisms mounted to a fluid transfer tip at the end of a fluid transfer hose. In FIG. 29a the lever member 934 is similar to that already described with respect to FIGS. 5 to 12, 13 to 15, and 19 to 27. In FIG. 29b, the lever member 1034 is similar to that described above with respect to FIGS. 16 to 18. Equally, such a hose or other fluid transfer device could replace the syringe shown in any of the other embodiments described above.

FIGS. 30a to 30e illustrate a further embodiment of a syringe 1102 that has a lever member 1134 pivotally mounted to the barrel 1104. In this embodiment it is optional for the lever member 1134 to be resiliently biased by a leaf spring or other spring means. Where a spring is not included, as is shown, the lever member 1134 can be manually moved between different pivotal positions. The lever member 1134 is provided with a thumb rest 1150 to ensure that a user has a good grip. The tapered tip 1106 of the syringe 1102 includes a gripping flange 1108 towards its lower end. The syringe 1102 may be connected to a hub 210 that is the same as previously shown in FIGS. 5c and 5d. In order to connect the hub 210 to the tip 1106, the lever member 1134 is placed in a neutral position as seen in FIGS. 30a and 30b. The cross-section of FIG. 30b shows that there is space surrounding the tip 1106 so that the hub 210 can be pushed down to engage its inner groove 216 over the flange 1108. Once the hub 210 is connected in its locked position, seen in FIG. 30c, the lever member 1134 can be pulled to one side so that the lower half of the slot 1139, which has a smaller diameter, presses tightly around the hub 210 and assists in holding the hub 210 on the tip 1106. When it is desired to disconnect the hub 210 from the tip 1106, a user pivots the lever member 1134 to its opposite position, as is shown in FIGS. 30d and 30e. In this position the upper end of the slot 1139, which has a larger diameter, surrounds the tip so that it is no longer holding the flange and groove in locking engagement. Furthermore, as the lever member 1134 is pivoted its front surface 1138 moves forward along the tip so as to push the hub 210 out of the locked position, and subsequently to release the hub 210 from its friction fitting. Although the lever member 1134 is not shown as including a catch member, a catching mechanism as previously described may optionally be added. It is also envisaged that one or more spring members may be used to bias movement of the lever member 1134.

It will be appreciated that various different hub designs have been described above. In FIGS. 5c and 5d the hub 210 is shown as having a groove 216 on its inner surface to engage over a corresponding flange when it is connected to a tip. In an alternative embodiment seen in FIGS. 31a and 31b, the hub 210′ has a flange 216′ on its inner surface rather than a groove. A corresponding tip may be grooved for gripping engagement with the flange 216′. Furthermore, in FIGS. 5c and 5d the hub 210 includes a continuous skirt 214. The hub 210 is moulded from a plastics material and therefore the skirt 214 has a degree of elasticity allowing it to flex. Depending on the rigidity of the plastics material, the skirt may be made more flexible by splitting it into separate segments. FIGS. 32a and 32b show a first version of a hub 610 where the skirt 614 is segmented. FIGS. 33a and 33b show a second version of a hub 710 where the skirt 714 has segments cut at different circumferential positions.

FIGS. 34 and 35 compare the hub 210 previously seen in FIGS. 5c and 5d with conventional hubs. FIGS. 34a and 35a show the hub 210. FIGS. 34b and 35b show a standard Luer lock hub 810 having an outer thread 812 but no skirt. FIGS. 34c and 35c show a standard Luer slip hub 910 having an outer rim 912 but no skirt.

Although the present invention has been described in the context of syringe of other fluid transfer device having a “male” connector tip that is externally, tapered to form a friction fit when inserted in a corresponding “female” hub, the various disconnecting mechanisms outlined above may equally find use in releasing the connection between a “female” connector tip and a “male” hub. FIG. 36 shows an alternative syringe 1202 with a female connector tip 1206 that is internally tapered to form a friction fit when a corresponding male hub 1210 is inserted therein. It will be appreciated that such a design deviates from the standard design of a Luer slip connection but the principles of releasing the friction fit are the same and hence encompassed within the scope of the present invention. Such a reversal of the hub design may be particularly applicable to containers for blood collection tubes.

While the invention has been described in the context of various embodiments, these are merely examples and features of one embodiment may be combined with those of another and vice versa. Furthermore, although not illustrated in all of the embodiments, a locking or blocking member may be provided to prevent the disconnecting lever, sleeve, etc. from moving out of its first position until it is desired to be able to operate the disconnecting mechanism. The disconnecting mechanism could be disabled, for example, by a blocking interaction with a cap on the syringe needle (where provided). Further variants and suitable features will be apparent to the skilled person. The scope of the invention is defined by the following claims.

We claim:

1. A fluid transfer device, comprising:
a fluid transfer tip, the fluid transfer tip comprising a tapered friction fitting for a corresponding hub;
an additional means for gripping a hub in a locked position when connected to the fluid transfer tip by the friction fitting; and
a disconnecting member provided with a resilient bias that causes it to engage the gripping means and thereby assist in holding the hub in the locked position;
wherein the disconnecting member is moveable relative to the fluid transfer tip and against the resilient bias so as to release engagement of the gripping means, move the hub out of the locked position provided by the gripping means and release the hub from the friction fitting.

2. A device according to claim 1, wherein the disconnecting member comprises at least one lever member pivotally mounted to the device.

3. A device according to claim 2, wherein the lever member comprises a front surface that is substantially transverse to the axis of the tip and one or more side surfaces that extend in a direction substantially parallel to the axis of the tip.

4. A device according to claim 2, wherein the front surface is curved such that initial movement of the lever member against the resilient bias moves the front surface substantially transverse to the fluid transfer tip to release engagement of the gripping means and further movement of the lever member against the resilient bias moves the front surface along the fluid transfer tip to move the hub out of the locked position provided by the gripping means and release the hub from the friction fitting.

5. A device according to claim 1, further comprising a catch means arranged to catch the hub after it has been released from the friction fitting.

6. A device according to claim 5, wherein further movement of the disconnecting member against the resilient bias causes the catch means to catch the hub.

7. A device according to claim 5, wherein the catch means is arranged to be released by resiliently biased movement of the disconnecting member.

8. A device according to claim 1, wherein the additional means for gripping a hub comprises a flange or groove provided on the fluid transfer tip.

9. A device according to claim 8, wherein the disconnecting member has a surface that is resiliently biased to at least partly surround the flange or groove and thereby assist in holding the hub in the locked position.

10. A device according to claim 8, wherein the disconnecting member has a surface that varies in diameter so as to provide a tight fit surrounding the flange or groove only when the disconnecting member is moved by the resilient bias.

11. A device according to claim 1, wherein the additional means for gripping a hub comprises one or more fingers arranged to grip onto an outer or inner surface of the hub.

12. A device according to claim 11, wherein the disconnecting member has one or more supports aligned with respective fingers and the disconnecting member is resiliently biased to press the support(s) against the finger(s) in the locked position.

13. A device according to claim 12, wherein the disconnecting member is moveable against the resilient bias to release the support(s) and thereby release the fingers.

14. A device according to claim 13, wherein the disconnecting member is further moveable against the resilient bias to push the fingers away from the hub.

15. A device according to claim 11, wherein the fingers comprise one or more gripping teeth.

16. A device according to claim 11, wherein the fingers comprise at least one thread arranged to engage with a thread on the hub.

17. A device according to claim 11, wherein the fingers comprise a stepped gripping surface.

18. A device according to claim 1, wherein the additional means for gripping a hub comprises a latch mechanism at the base of the fluid transfer tip.

19. A device according to claim 18, wherein the latch mechanism comprises one or more snap-fit members.

20. A device according to claim 18, wherein the disconnecting member has a surface that is resiliently biased to bear on the latch mechanism and thereby assist in holding the hub in the locked position.

21. A device according to claim 18, wherein the disconnecting member has a surface that varies in diameter so as to bear on the latch mechanism only when the disconnecting member is moved by the resilient bias.

22. A device according to claim 1, wherein the disconnecting member comprises at least one latch member arranged to engage with the hub and assist in holding the hub in the locked position.

23. A device according to claim 22, wherein movement of the disconnecting member against the resilient bias disengages the at least one latch member from the hub.

24. A device according to claim 22, wherein the at least one latch member engages with an inside or outside surface of the hub.

25. A device according to claim 1, wherein the disconnecting member is provided with a second resilient bias acting to oppose the first resilient bias so that an intermediate position can be achieved.

26. A device according to claim 1, wherein the disconnecting member comprises a surface that is arranged to be acted upon by a hub as it is connected to the fluid transfer tip such that the disconnecting member is temporarily moved against the resilient bias to accommodate the hub.

27. A device according to claim 26, wherein the disconnecting member comprises a slot and the fluid transfer tip passes through the slot, the slot providing space around the tip to accommodate the hub when the disconnecting member is temporarily moved against the resilient bias.

28. A device according to claim 27, wherein the surface arranged to be acted upon by a hub is provided by the slot.

29. A device according to claim 26, wherein the surface arranged to be acted upon by a hub is provided by a nose extending substantially parallel to the fluid transfer tip.

30. A device according to claim 29, wherein the disconnecting member comprises a nose extending substantially parallel to the fluid transfer tip and having an angled forward surface that is arranged to be acted upon by a hub as it is connected to the fluid transfer tip.

31. A device according to claim 26, wherein the disconnecting member is moved by the resilient bias to engage the gripping means after the hub has been accommodated and connected to the tip.

32. A device according to claim 1, further comprising means to lock the disconnecting member against the resilient bias.

33. A fluid transfer device, comprising:
a fluid transfer tip, the fluid transfer tip comprising a tapered friction fitting for a corresponding hub;
a lever member pivotally mounted to move relative the fluid transfer tip; and
a screw thread mounted on the lever member to enable a hub to be connected to the tip by a screw fit in addition to the friction fitting;
wherein the lever member is resiliently biased so that the screw thread is positioned to form a screw fit with the hub;
and wherein the hub can be disconnected by pivoting the lever member against the resilient bias to release the screw fit with the hub and to subsequently release the hub from the friction fitting.

34. A device according to claim 33, further comprising fingers that have a thread to additionally grip the hub when the lever member is resiliently biased to form a screw fit.

35. A device according to claim 34, wherein the fingers are arranged to disengage from the hub by the lever member pivoting against the resilient bias.

36. A device according to claim 33, wherein the lever member comprises a front surface that is substantially transverse to the axis of the tip and the front surface is arranged to move along the tip when the lever member is pivoted against the resilient bias.

37. A fluid transfer device, comprising:
a fluid transfer tip, the fluid transfer tip comprising a tapered friction fitting for a corresponding hub;
an additional means for gripping a hub in a locked position when connected to the fluid transfer tip by the friction fitting; and
a disconnecting member comprising an actuator portion and operable to move the actuator portion between at least two different positions, wherein in a first position the actuator portion engages the gripping means and thereby assists in holding the hub in the locked position and in a second position the actuator portion does not engage the gripping means but acts to move the hub out of the locked position and release the hub from the friction fitting.

38. A device according to claim 37, wherein the disconnecting member is manually operable.

39. A device according to claim 37, wherein the disconnecting member is provided with a resilient bias that moves the actuator portion into the first position.

40. A device according to claim 37, wherein the disconnecting member is operable to move the actuator portion into a third position, between the first and second positions, where the actuator portion releases the gripping means and allows a hub to be connected to the fluid transfer tip by the friction fitting.

41. A device according to claim 37, wherein the actuator portion extends in a plane substantially transverse to the axis of the fluid transfer tip.

42. A device according to claim 41, wherein the actuator portion is arranged to move along the tip when the disconnecting member is moved between the different positions.

43. A device according to claim 37, wherein the disconnecting member comprises one of more lever members.

44. A device according to claim 1, wherein the fluid transfer tip comprises a male connector tip that is tapered to form a friction fit when inserted in a corresponding female hub.

45. A device according to claim 1, wherein the fluid transfer tip comprises a tapered female part that forms a friction fit when a corresponding male part provided by the hub is inserted therein.

46. A fluid transfer device according to claim 1, wherein a hub is connected to the fluid transfer tip at least by the friction fitting.

47. A device according to claim 46, wherein the hub comprises a tapered internal surface and an outer rim at its base.

48. A device according to claim 46, wherein the hub comprises a tapered internal surface and an outer thread at its base.

49. A device according to claim 46, wherein the hub comprises a tapered internal surface and an outer rim or thread spaced from its base by a skirt portion.

50. A hub for directing fluid from a fluid transfer device, the hub comprising a tapered internal surface to form a friction fit with a corresponding fluid transfer tip and an outer rim or thread to enable it to be positively engaged when connected to a fluid transfer tip by the friction fit, wherein the outer rim or thread is spaced from a base of the hub by a skirt portion.

51. A hub according to claim 50, further comprising an additional means for gripping a fluid transfer tip when the hub is connected thereto by the friction fit.

52. A hub for directing fluid from a fluid transfer device, the hub comprising a tapered surface to form a friction fit with a corresponding fluid transfer tip, a threaded collar or rim external to the tapered surface, and an additional means for gripping a fluid transfer tip when the hub is connected thereto by the friction fit.

53. A hub according to claim 51, wherein the additional means for gripping a tip comprises a flange or groove provided on the tapered surface.

54. A hub according to claim 52, wherein the threaded collar or rim is spaced from a base of the hub by a skirt portion.

55. A hub according to claim 49, wherein the skirt portion is flexible.

56. A hub according to claim 50, connected to a fluid transfer tip provided by a fluid transfer device in the form of a syringe, blood collection tube or hose.

57. A device according to claim 1, wherein the hub comprises a cannula or hypodermic needle and the fluid transfer device comprises a syringe.

58. A fluid transfer device comprising:
a fluid chamber in communication with a male connector tip, the male tip being tapered to form a friction fit when inserted in a corresponding female hub;
a disconnecting member moveable relative to the male connector tip between a first position proximal to the fluid chamber and a second position spaced from the first position towards a distal end of the male connector tip; and
means for gripping a hub when connected to the tip in use.

59. A device according to claim 58, wherein the disconnecting member is arranged to release the gripping means when moving between the first and second positions.

60. A device according to claim 58, wherein the gripping means operates automatically when the tip is connected to a corresponding hub.

61. A device according to claim 58, wherein the gripping means comprises one or more latch members or snap-fit members.

62. A device according to claim 58, wherein the disconnecting member comprises a lever member pivotally connected to the device with a front surface moveable between the first and second positions relative to the male connector tip.
